(19) United States
(12) Patent Application Publication
(10) Pub. No.: US 2006/0281966 A1
(43) Pub. Date: Dec. 14, 2006

(54) Aneurysm treatment system and method

(76) Inventor: James C. Peacock III, San Carlos, CA (US)

Correspondence Address: JOHN P. O'BANION;O'BANION & RITCHEY LLP, 400 CAPITOL MALL SUITE 1550, SACRAMENTO, CA, 95814, US

(21) Appl. No.: 11/404,374

(22) Filed: Apr. 14, 2006

Related U.S. Application Data
(63) Continuation of application No. PCT/US04/34106, filed on Oct. 14, 2004.
(60) Provisional application No. 60/511,170, filed on Oct. 14, 2003.

(51) Int. Cl. A61F 2/94; A61F 2/06

(52) U.S. Cl. 600/37; 623/1.13

(57) ABSTRACT

An external aneurysm support scaffold is implanted around an exterior surface of an aneurysm and prevents substantial dilation or progression of the AAA. Minimally invasive delivery is used via port-access, i.e. for aortic aneurysms along the back, abdomen, or thorax, to a location externally adjacent the aneurysm, such as via laparascopic delivery. The scaffold is unwound or unfolded in-situ to extend partially (e.g. about 270 degrees) or completely circumferentially around the aneurysm. Unique delivery devices allow for deployment around the aneurysm. Gaps between an array of transverse fingers of the scaffold may accommodate branch vessels extending from the aneurismal vessel, such as aortic perforators. An agent is injected to treat an aneurysm, such as by providing support, cell retention or recruitment, and/or angiogenesis. Living cells are delivered to treat an aneurysm. An adjustable graft polymerizes in-situ to support an aneurysm conformed therewith.

CROSS-REFERENCE TO RELATED APPLICATIONS

This application claims priority from, and is a 35 U.S.C. § 111(a) continuation of, co-pending PCT international application serial number PCT/US2004/034106, filed on Oct. 14, 2004, incorporated herein by reference in its entirety, which designates the U.S., which claims priority from U.S. Provisional Patent Application Ser. No. 60/511,170, filed on Oct. 14, 2003, which is herein incorporated in its entirety by reference thereto.

### BACKGROUND OF THE INVENTION

1. Field of the Invention

This invention relates to the field of medical devices, and more particularly to systems and methods for treating aneurysms in the body, and still more particularly for treating vascular aneurysms, and still more specifically for treating thoracic and abdominal aortic aneurysms.

2. Description of Related Art

Abdominal aortic aneurysms (“AAA”) are a significant medical problem that often may lead to death if left untreated and in the event of rupture.

Substantial efforts have been expended to provide therapies for this condition.

One series of therapies are direct surgery. Another series of therapies include percutaneous translumenal delivery of endo-aortic stent grafts to the region of the AAA to isolate the compromised aneurysmic wall from harmful endo-aortic blood pressures as an inside-out approach.

The direct surgical efforts are major medical undertakings, and are correlated with substantial patient morbidity, long times in the OR, high costs, and still high incidence of ongoing problems. The percutaneous translumenal endo-aortic grafting measures involve very large implants within the most major artery of the body, and also relate to high patient morbidity associated with introduction cut-downs in the legs. In particular, the tremendous size of the stent-grafts themselves, even when “folded” during delivery and prior to expansion within a AAA, requires substantial size for delivery or introducer sheaths, thus not typically allowing for “Seldinger” technique of vascular access which would be better desired with lower morbidity to such cut-downs.

According to the substantial shortcomings of existing procedures, both surgical and percutaneous, many early incidences of AAA are left untreated, as the cure is often considered worse than the solution. Watchful waiting becomes the lifestyle-of such patients, and their healthcare providers, that would otherwise be considered lucky for catching a potential deadly AAA early. Once the AAA progresses to critical dilation, only then is an invasive procedure undertaken.

It is to be appreciated that the foregoing shortcomings of conventional, and even more contemporary, MA therapies have similar import to treatment of other types of aneurysms, and in particular without limitation with respect to thoracic aortic aneurysms.

There is still a need for a less-invasive or minimally invasive solution to treating aneurysms, and in particular vascular aneurysms, still more particularly aortic aneurysms, and still further in particular thoracic or abdominal aortic aneurysms.

There is also still a need for a system and method that provides acceptable therapy for early diagnosed aneurysms, and in particular vascular aneurysms, still more particularly aortic aneurysms, and still further in particular thoracic or abdominal aortic aneurysms.

There is also still a need for a system and method that provides acceptable prophylaxis of aneurysm progression in early diagnosed aneurysms, and in particular vascular aneurysms, still more particularly aortic aneurysms, and still further in particular thoracic or abdominal aortic aneurysms.

There is also still a need for a system and method that provides a medically acceptable outside-in approach to treating aneurysms, and in particular vascular aneurysms, still more particularly aortic aneurysms, and still further in particular thoracic or abdominal aortic aneurysms.

There is also still a need for improved systems and methods for treating aneurysms with improved patient morbidity, and in particular for treating vascular aneurysms, still more particularly aortic aneurysms, and still further in particular thoracic or abdominal aortic aneurysms.

There is also still a need for improved systems and methods for treating aneurysms earlier such that the issues associated with the interventional procedure do not outweigh the issues associated with the early detected aneurysm. This need exists in particular with respect to vascular aneurysms, still more particularly aortic aneurysms, and still further in particular thoracic or abdominal aortic aneurysms.

There is also still a need for improved systems and methods for treating aneurysms with reduced costs. This need exists in particular with respect to vascular aneurysms, still more particularly aortic aneurysms, and still further in particular thoracic or abdominal aortic aneurysms.

There is also still a need for improved systems and methods for treating aneurysms more quickly and easily than prior efforts. This need exists in particular with respect to vascular aneurysms, still more particularly aortic aneurysms, and still further in particular thoracic or abdominal aortic aneurysms.

### BRIEF SUMMARY OF THE INVENTION

The invention therefore provides various aspects that are considered generally beneficial over prior disclosures and commercial efforts to treat aneurysms, and in particular vascular aneurysms, still more particularly aortic aneurysms, and still further in particular thoracic or abdominal aortic aneurysms.

One aspect of the invention provides a minimally invasive system and related method adapted to treat aneurysms, and in particular vascular aneurysms, still more particularly aortic aneurysms, and still further in particular thoracic or abdominal aortic aneurysms.

Another aspect of the invention is a system and method that is adapted to provide substantial therapy for early diagnosed aneurysms, and in particular vascular aneurysms, still more particularly aortic aneurysms, and still further in particular thoracic or abdominal aortic aneurysms.

Another aspect of the invention is a system and method that is adapted to provide substantial prophylaxis of aneurysm progression in early diagnosed aneurysms, and in particular vascular aneurysms, still more particularly aortic aneurysms, and still further in particular thoracic or abdominal aortic aneurysms.

Another aspect of the invention is a system and method that is adapted to provide an outside-in approach to treating aneurysms, and in particular vascular aneurysms, still more particularly aortic aneurysms, and still further in particular thoracic or abdominal aortic aneurysms.

Another aspect of the invention is a system and method that is adapted to treat aneurysms with improved patient morbidity versus previously disclosed percutaneous or other translumenal stent-graft systems and methods. According to one particular beneficial mode, the system and method is adapted to provide improved patient morbidity associated with therapy of vascular aneurysms, still more particularly aortic aneurysms, and still further in particular thoracic or abdominal aortic aneurysms.

Another aspect of the invention is a system and method that is adapted to treat aneurysms earlier than available using previously disclosed percutaneous or other translumenal stent-graft approaches, such that the risks associated with the interventional procedure do not outweigh the risks associated with the early detected aneurysm. This system is in particular well adapted for use in treating vascular aneurysms, still more particularly aortic aneurysms, and still further in particular thoracic or abdominal aortic aneurysms.

Another aspect of the invention is a system and method that is adapted to treat aneurysms with reduced costs when compared with conventional percutaneous or other translumenal stent-graft approaches. This system is in particular well adapted for use with respect to vascular aneurysms, still more particularly aortic aneurysms, and still further in particular thoracic or abdominal aortic aneurysms.

Another aspect of the invention is a system and method that is adapted to treat aneurysms more quickly and easily than available with previously disclosed percutaneous or other translumenal stent-graft approaches. This system and method is in particular well suited for use with respect to vascular aneurysms, still more particularly aortic aneurysms, and still further in particular thoracic or abdominal aortic aneurysms.

Another aspect of the invention is an abdominal aortic aneurysm (AAA) support system that includes a support scaffold agent and a delivery assembly that is adapted to deliver the support scaffold agent to a location adjacent to an exterior surface of an AAA. The system further includes means for providing an external support scaffold to the AAA with the support scaffold agent delivered to the location.

Another aspect of the invention is an abdominal aortic aneurysm (AAA) support system that includes a support scaffold agent and a delivery assembly. The support scaffold agent includes a mechanical support scaffold that is adjustable between a first configuration and a second configuration.

The delivery assembly is adapted to deliver the mechanical support scaffold in the first configuration to a location within a patient's body externally adjacent to a AAA. The mechanical support scaffold is adjustable at the location from the first configuration to the second configuration. In the second configuration at the location, the support scaffold agent is adapted to provide a substantial external support scaffold to the AAA.

Another aspect of the invention is an abdominal aortic aneurysm (AAA) support system with a support scaffold agent that includes a graft material that is adapted to be positioned, in a first condition that is substantially pliable, at a location around an exterior surface of an AAA and with a shape substantially conformed to the exterior surface of the AAA. The graft material at the location is adjustable from the first condition to a second condition that substantially holds the shape and is substantially less pliable than the first condition. In the second condition, the graft material provides a substantial external support scaffold to the AAA.

Another aspect of the invention is an abdominal aortic aneurysm (AAA) support system that includes a support scaffold agent that is adapted to be implanted at a location along an exterior surface of an AAA and to provide a substantial external support scaffold to the AAA.

Another aspect of the invention is an aneurysm support system that includes a support scaffold agent and a delivery assembly that is adapted to deliver the support scaffold agent to a location over an exterior surface of an aneurysm. This system further includes means for providing an external support scaffold to the aneurysm with the support scaffold agent delivered to the location.

Another aspect of the invention is an aneurysm support system that includes a support scaffold agent that is adjustable between a first configuration and a second configuration, and also includes means for delivering the support scaffold agent in the first configuration to a location within a patient's body externally adjacent to an aneurysm. The support scaffold agent is adjustable at the location from the first configuration to the second configuration. The support scaffold agent in the second configuration at the location is adapted to provide a substantial external support scaffold to the aneurysm.

Another aspect of the invention is an aneurysm therapy system that includes a graft material that is adapted to be positioned in a first condition that is substantially pliable at a location adjacent to an exterior surface of an aneurysm and with a shape conformed around the exterior surface of the aneurysm. The graft material is adjustable from the first condition to a second condition that provides a substantial therapeutic result to the aneurysm.

According to one mode of this aspect, the graft material is further adapted to form at least in part a support scaffold agent that provides a substantial support scaffold to the aneurysm.

Another aspect of the invention is an aneurysm support system that includes a support scaffold agent that is adapted to be implanted around an exterior surface of an aneurysm and to form an external support scaffold to the aneurysm.

Another aspect of the invention is an aneurysm support system with a delivery assembly and a support scaffold agent that comprises a polymer agent coupled to the delivery assembly and that is adapted to be delivered to a surface of an aneurysm with the delivery assembly and to form a support scaffold to the aneurysm.

Another aspect of the invention is an aneurysm support system with a delivery assembly and an injectable agent coupled to the delivery assembly.

The delivery assembly is adapted to deliver the injectable agent to a location along a wall of an aneurysm. The injectable agent when delivered to the location is adapted to promote, to an extent sufficient to provide a substantial therapeutic effect to the aneurysm at the location, at least one of: retention of cells delivered to the location, cellular recruitment at the location, or angiogenesis at the location.

According to one mode of this aspect, the injectable agent is further adapted to form at least in part a support scaffold agent that provides a substantial support scaffold to the aneurysm.

Additional modes, embodiments, variations, and features of the foregoing aspects and modes are further provided as follows.

According to aspects and modes providing a support scaffold agent, one further mode provides the support scaffold agent to include an adjustable mechanical scaffold wall.

According to one embodiment of this mode, the adjustable mechanical scaffold wall comprises an adjustable stent scaffold. In a further embodiment, the mechanical scaffold wall further comprises a graft member coupled to the adjustable stent scaffold to form an adjustable stent-graft composite member.

According to another embodiment, the mechanical scaffold wall includes first and second opposite longitudinal ends along a longitudinal axis, and first and second opposite transverse ends transverse to the longitudinal axis. The mechanical scaffold wall is adjustable from a first configuration having a first shape with a first diameter to a second configuration having a shape with a second diameter. The second shape comprises a memory shape for the mechanical scaffold wall, whereas the first shape comprises a deformed shape for the mechanical scaffold wall. Accordingly, the mechanical scaffold wall is adjustable from the second configuration to the first configuration under an applied deflection force, and is adjustable from the first configuration to the second configuration under a memory recovery force at least in part upon removal of the applied deflection force. The first shape and first diameter are configured such that the mechanical scaffold wall is deliverable to the location through a delivery passageway of a delivery member. The second shape comprises a radius of curvature around the longitudinal axis with the first and second transverse ends extending around a circumference about the longitudinal axis and such that the second diameter is substantially larger than the first diameter, wherein the second shape is adapted to conformably engage an exterior surface of the aneurysm.

According to one variation of this embodiment, in the second configuration at the location the first and second transverse ends do not meet and the mechanical scaffold wall only partially circumferentially covers the aneurysm.

According to another variation, in the second configuration at the location the mechanical scaffold wall is adapted to completely circumferentially surround the aneurysm. One feature that may be included for this variation provides that, in the second configuration at the location, at least one of the first and second transverse ends is adapted to be secured to a portion of the mechanical scaffold wall at or adjacent to the other of the first and second transverse ends. Further to this feature, a securing assembly may also be provided that is adapted to secure the first and second transverse ends relative to each other in the second configuration at the location.

According to another embodiment, the mechanical scaffold wall comprises a shape memory material. In another embodiment, the mechanical scaffold wall comprises a superelastic alloy material. According to either or both of these embodiments, the mechanical scaffold wall may be constructed at least in part from a nickel-titanium alloy material.

In another embodiment, the mechanical scaffold wall includes a spine extending between the first and second longitudinal end portions along a longitudinal axis. Also included in the wall is a plurality of transverse ribs extending from the spine transverse to the longitudinal axis and separated by gaps.

According to one variation of this embodiment, the spine may located along a first transverse end of the mechanical scaffold wall, and the plurality of transverse ribs extends transversely from the spine and from the first transverse end to a second transverse end. In another variation, the spine is located along an intermediate region between two transverse ends of the mechanical scaffold wall, and two transverse arrays of ribs extend therefrom in opposite directions toward two transverse ends of the wall. A first transverse array of ribs extends from the spine transversely toward the first end. A second transverse array of ribs extends from the spine transversely toward the second end.

According to a further variation, the gaps between ribs are adapted to receive side branch vessels when the mechanical scaffold wall is adjusted to the second configuration around the aneurysm.

In another variation, the ribs comprise substantially flat planar members with a width between gaps and a length transverse to the longitudinal axis with a secondary shape around a radius of curvature around the longitudinal axis.

In still a further variation, the ribs include elongated loop-shaped members with voids extending between adjacent spline members extending transverse to the longitudinal axis.

In yet another variation, the ribs each comprise at least one elongated, shaped spline extending from the spine and undulating with a tertiary shape along an axis transverse to the longitudinal axis.

In still another variation, the spine and transverse ribs comprise a skeletal stent member, and the mechanical scaffold wall further includes a graft member coupled to the skeletal stent member.

According to another mode variously applicable to the foregoing aspects, a delivery assembly is provided that is adapted to deliver the respective active components of the respective system, such as a support scaffold agent, graft assembly, injectable agent, etc.

In one highly beneficial embodiment of this mode, the delivery assembly further comprises a minimally invasive introducer sheath.

In another beneficial embodiment, the delivery assembly is adapted to provide access to the location along a AAA from an anterior location along the patient in a transperitoneal delivery approach.

In another embodiment, the delivery assembly further includes a laparoscopic delivery assembly with a laparoscope.

In another embodiment, the delivery assembly is adapted to provide access to the location along a AAA from a posterior location adjacent the spine.

In another embodiment, the delivery assembly is adapted to provide access to the location along a AAA from an axial location along a side of the patient.

In another embodiment related to aspects and modes providing a support scaffold agent to aneurysms, the support scaffold agent comprises a mechanical scaffold wall that extends between first and second longitudinal ends along a longitudinal axis and between first and second transverse ends along a transverse axis that is transverse to the longitudinal axis. The mechanical scaffold wall is adjustable between a wound configuration, which is wound around the longitudinal axis with the first transverse end located on an exterior radial surface of the wound wall and with the second transverse end located interiorly of the wound wall, and an unwound configuration. The delivery assembly in a first condition is adapted to retain the mechanical scaffold wall in the wound configuration for delivery to a deployment location along the exterior of the aneurysm. The delivery assembly is adjustable to a second condition at the deployment location that is adapted to unwind the mechanical scaffold wall to the unwound configuration over the aneurysm.

Various further embodiments related to the foregoing embodiment are also provided as follows.

According to one such further embodiment, the unwound configuration is characterized as a memory condition for the mechanical scaffold wall, whereas the wound configuration is characterized as a deformed condition for the mechanical scaffold wall. Accordingly, the mechanical scaffold wall is adjustable from the unwound configuration to the wound configuration under an applied force, and is adjustable from the wound configuration to the unwound configuration by memory recovery force at least in part upon removal of the applied force. The delivery assembly comprises a delivery catheter with a tubular wall that defines a delivery lumen and a longitudinal slot therethrough the tubular wall. The mechanical scaffold wall is held within the delivery lumen in the wound configuration under radial retention force.

The delivery assembly is adapted to unwind the mechanical scaffold wall to the unwound configuration at least in part by extending the first transverse end from the delivery lumen through the slot.

According to one variation of this further embodiment, an adjustable lock assembly is provided that is adapted to selectively release or retain the mechanical scaffold wall respectively from or in the wound configuration.

According to another variation, an internal control assembly is provided that is coupled to the mechanical scaffold wall within the delivery lumen and that is adapted to at least in part control deployment of the mechanical scaffold wall to the unwound configuration at the location around the aneurysm. The internal control assembly may be further adapted to retract the mechanical scaffold wall back into the delivery lumen from a fully or partially deployed unwound configuration at the location, according to a further feature.

According to another further embodiment, the wound configuration is characterized as a memory condition for the mechanical scaffold wall, whereas the unwound configuration is characterized as a deformed condition for the mechanical scaffold wall. Accordingly, the mechanical scaffold wall is adjustable from the wound configuration to the unwound configuration under an applied force, and is adjustable from the wound configuration to the unwound configuration by memory recovery force at least in part upon removal of the applied force. The delivery assembly includes a substantially torqueable delivery member that is rotatable around the longitudinal axis and is selectively engaged with the second transverse end within the wound configuration of the mechanical scaffold wall. The delivery assembly is adapted to unwind the mechanical scaffold wall to the unwound configuration over the aneurysm upon rotation of the delivery member and deflection of the first transverse end away from the longitudinal axis and over the adjacent aneurysm wall. Further included with this system may be a means for deflecting the first transverse end over the aneurysm. Also included may be an adjustable lock assembly associated with the delivery member and that is adapted to selectively retain or release the second transverse end of the mechanical scaffold wall respectively from or in the wound configuration.

According to another mode related to foregoing aspects and modes providing a support scaffold agent, the support scaffold agent may include a mechanical scaffold wall that is adjustable between a folded configuration, which is folded relative to a longitudinal axis, and an unfolded configuration.

In this mode, however, the unfolded configuration is characterized as a memory condition for the mechanical scaffold wall, and the folded configuration is characterized as a deformed condition for the mechanical scaffold wall. Accordingly, the mechanical scaffold wall is adjustable from the unfolded configuration to the folded configuration under an applied retention force, and is adjustable from the folded configuration to the unfolded configuration by memory recovery force at least in part upon removal of the applied radial retention force. The delivery assembly includes a delivery catheter with a tubular wall that defines a delivery lumen and a first longitudinal slot therethrough the tubular wall. The delivery catheter in a first condition is adapted to hold the mechanical scaffold wall in the folded configuration under radial retention force for delivery to a deployment location exteriorly adjacent to the aneurysm. The delivery catheter is adjustable at the deployment location to a second condition that is adapted to release the mechanical scaffold wall from radial retention to the unfolded configuration around the aneurysm at least in part by allowing the first transverse end to extend from the delivery lumen through the first longitudinal slot.

According to one embodiment of this mode, the delivery catheter further includes a second longitudinal slot therethrough the tubular wall. The delivery catheter in the second condition is adapted to release the mechanical scaffold wall from radial retention to the unfolded configuration around the aneurysm at least in part by further allowing the second transverse end to extend from the delivery lumen through the second longitudinal slot.

According to one variation of this embodiment, the delivery assembly is adapted to deliver the mechanical support wall to a deployment location that is at an anterior position relative to the aneurysm. In the unfolded configuration, the mechanical support wall has a curved shape with the first and second transverse ends curving around opposite transverse sides of the aneurysm.

In another variation, first and second adjustable lock assemblies are provided that are adapted to selectively release or retain the mechanical scaffold wall from extending radially through the first and second longitudinal slots, respectively.

In still another variation, an internal control assembly is provided that is coupled to the mechanical scaffold wall within the delivery lumen and that is adapted to at least in part control deployment of the mechanical scaffold wall to the unfolded configuration at the location around the aneurysm. According to one feature that may be provided under this variation, the internal control assembly may be further adapted to retract the mechanical scaffold wall back into the delivery lumen from a fully or partially deployed unfolded configuration at the location.

According to another further mode of the foregoing aspects and modes, the various active therapeutic components, e.g. support scaffold agent, graft assembly and/or material, or injectable agent, is provided to include a polymer agent.

In one variation of this mode, the polymer agent is a synthetic polymer agent.

In another variation, the polymer agent includes polyethylene oxide (“PEO”), or an analog, derivative, precursor, or agent thereof.

In another variation, the polymer agent includes PEO-poly-l-lactic acid (“PLLA-PEO block copolymer”), or an analog, derivative, precursor, or agent thereof.

In another variation, the polymer agent includes poly (N-isopropylacrylamide-co-acrylic acid) (“poly(NIPAAm-co-Aac)”), or an analog, derivative, precursor, or agent thereof.

In another variation, the polymer agent includes a pluronic agent, or an analog, derivative, or precursor thereof.

In another variation, the polymer agent includes poly-(N-vinyl-2-pyrrlidone (“PVP”), or an analog, derivative, precursor, or agent thereof.

In another variation, the polymer agent includes a biologic polymer agent.

In another variation, the polymer agent includes alginate, or an analog, derivative, precursor, or agent thereof.

In another variation, the polymer agent includes a block polysaccharide, or an analog, derivative, precursor, or agent thereof.

In another variation, the polymer agent includes collagen, or an analog, derivative, precursor, or agent thereof.

In another variation, the polymer agent includes a fibrin glue agent, or an analog, derivative, precursor, or agent thereof.

In another variation, the polymer agent is adapted to promote angiogenesis at the location along the aneurysm.

In another variation, the polymer agent is adapted to promote cellular recruitment at the location along the aneurysm.

In another variation, the system further includes a volume of living cells that are adapted to be delivered in combination with the polymer agent to the location. The living cells may include, in highly beneficial examples, myoblasts, fibroblasts, stem cells, or endothelial or EPC cells. The living cells may be foreign, or in a highly beneficial feature may include autologous cells.

In still a further feature, the living cells may include genetically modified cells.

In another variation, the living cells provided with the polymer agent are adapted to be coupled to and delivered to the location via a common delivery member as the polymer agent.

In another polymer agent variation, the polymer agent is adapted to enhance retention of the living cells at the location. In another variation, the polymer agent is injectable.

In addition to providing a polymer agent, additional further embodiments also provide in combination therewith an adjustable mechanical scaffold wall that is adapted to provide external scaffold support to the aneurysm.

According to another mode related to the foregoing aspects and modes providing a support scaffold agent, the support scaffold agent is adapted to provide external scaffold support in particular to a AAA.

In yet another related mode, the support scaffold agent is adapted to provide external scaffold support to a cerebral aneurysm.

In still another related mode, the support scaffold agent is adapted to provide external scaffold support to a thoracic aortic aneurysm.

Another aspect of the invention is a tissue wall therapy system that includes a graft material that is adapted to be positioned in a first condition that is substantially pliable at a location adjacent to a surface of tissue wall structure in a body of a patient and with a shape conformed around the surface of the tissue wall structure. The graft material is adjustable at the location from the first condition to a second condition that is implantable at the location, is substantially less pliable than the first condition, and provides a substantial therapeutic result to the tissue wall structure.

Another aspect of the invention is a tissue wall therapy system that includes a graft material that is adjustable between first and second conditions and that comprises a graft wall, a first precursor agent coupled to the graft wall, and a second precursor agent coupled to the graft wall. In the first condition, the first and second precursor agents are relatively isolated from each other. In the second condition, the first and second precursor agents are substantially combined and reacted together.

According to one further mode related to the foregoing aspects and modes providing in particular a therapeutic graft assembly or material, the graft material comprises a graft wall, a first precursor agent coupled to the graft wall, and a second precursor agent coupled to the graft wall. In the first condition the first and second precursor agents are relatively isolated from each other. In the second condition, the first and second precursor agents are substantially combined and reacted together.

In one embodiment of this further mode, the graft wall includes a first plurality of reservoirs and a second plurality of reservoirs. The first precursor agent is located within the first plurality of reservoirs. The second precursor agent is located within the second plurality of reservoirs. In the first condition, the first and second pluralities of reservoirs are substantially isolated and do not communicate. In the second condition, the first and second pluralities of reservoirs communicate to allow mixing of the first and second precursor agents.

In one variation of this embodiment, an erodable material is located between the first and second pluralities of reservoirs. The graft material is adjustable from the first condition to the second condition by eroding the erodable material. In one further variation, the erodable material is erodable upon an applied energy, such as for example by being ultrasonically erodable.

In another further variation, the erodable material is bioerodable. In still another variation, the erodable material is chemically erodable.

According to a further embodiment, the first and second precursor materials are adapted to polymerize to provide a polymeric scaffold when mixed.

In another embodiment, the first and second precursor materials comprise thrombin and fibrinogen, respectively.

In still another further mode of the foregoing graft aspects and modes, the graft material or assembly in the second condition comprises a substantially polymerized polymer agent that is not included in the graft material in the first condition.

In another such further mode, the graft material in the second condition is adapted to provide substantial external scaffold support to the aneurysm.

According to a further mode related to foregoing aspects and modes providing an injectable agent, the injectable agent is in particular characterized as being a type that is adapted to promote cellular retention of cells delivered to the location.

In another such related further mode, the injectable agent is characterized as being a type that is adapted to promote recruitment of endogenous cells at the location.

In another such related further mode, the injectable agent is characterized as being a type that is adapted to promote angiogenesis at the location.

In another such related further mode, the injectable agent includes first and second precursor agents that are adapted to react when combined at the location. According to one embodiment of this further mode, the first and second precursor materials are adapted to polymerize when combined at the location. In a further more particular embodiment considered highly beneficial, the first and second precursor materials comprise fibrinogen and thrombin, respectively.

In a further mode related to the foregoing aspects and modes, bioactive agents such as drugs and the like may also be delivered in conjunction with, or principally as, the delivered active component described (e.g. injectable agent, etc.). In one highly beneficial example, an angiogenenic agent may be delivered to the location of therapy to further enhance vessel wall function. According to one highly beneficial embodiment, such an injectable agent may include pleiotrophin, or an analog, derivative, precursor, or agent thereof.

Further to the various foregoing support scaffold agent aspects and modes, in a further related mode the support scaffold agent is adapted to provide external scaffold support to the aneurysm sufficient to reduce dilation or progression of the aneurysm.

Additional aspects of the invention include various highly beneficial methods, including without limitation those methods related to assembly or use of the foregoing system aspects, modes, embodiments, variations, and features. Certain such additional aspects are described as follows, and constitute highly beneficial aspects of the invention.

One such method aspect provides a method for treating an abdominal aortic aneurysm (AAA) as follows. A minimally invasive introducer sheath with a delivery lumen is positioned within a body of a patient so as to provide transluminal access through the delivery lumen to a location within a patient's body that is externally adjacent to a AAA. A support scaffold agent is delivered in a first configuration through the delivery lumen of the minimally invasive introducer sheath to the location. The support scaffold agent is adjusted from the first configuration to a second configuration that is adapted to provide an external support scaffold to the AAA sufficient to provide a substantially therapeutic result to the AAA.

Another such aspect is a method for treating an abdominal aortic aneurysm (AAA) that includes delivering an injectable polymer agent onto a wall of an AAA.

Another method aspect of the invention is a method for treating an abdominal aortic aneurysm (AAA) by delivering living cells onto a wall of an AAA.

Another method aspect of the invention is a method for treating an abdominal aortic aneurysm (AAA) as follows. Living cells and a polymer agent are delivered to a common location adjacent to a wall of an AAA such that, in their combination together at the location, a therapeutic support scaffold is provided to the AAA.

Another method aspect of the invention treats an abdominal aortic aneurysm (AAA) by delivering an external support scaffold agent to an exterior surface of an AAA, and then providing a therapeutic external support scaffold to the AAA with the external support scaffold agent.

Another aspect of the invention is a method for treating an aneurysm as follows. A minimally invasive introducer sheath with a delivery lumen is positioned so as to provide translumenal access through the delivery lumen to a location within a patient's body that is externally adjacent to an aneurysm.

A support scaffold agent is delivered in a first configuration through the delivery lumen of the minimally invasive introducer sheath to the location.

The support scaffold agent is adjusted at the location from the first configuration to a second configuration that is adapted to provide a substantial external support scaffold to the aneurysm.

Another aspect of the invention is a method for treating an aneurysm by delivering a polymer agent onto a wall of the aneurysm such that the polymer agent provides a therapeutic effect to the aneurysm.

Another aspect of the invention is a method for treating an aneurysm by delivering living cells onto a wall of the aneurysm such that the living cells provide a therapeutic effect to the aneurysm.

Another aspect of the invention is a method for treating an aneurysm by delivering living cells and a polymer agent to a location along a wall of the aneurysm, such that the combination of the living cells and polymer agent delivered to the location together provide a therapeutic effect to the aneurysm.

Another aspect of the invention is a method for treating an aneurysm by providing an external support scaffold to a wall of the aneurysm sufficient to provide a substantial therapeutic result to the aneurysm.

Another aspect of the invention is a method for treating a tissue wall structure in a patient as follows. A graft assembly is delivered to a shaped surface of the tissue wall structure when the graft assembly is in a first condition. The graft assembly is adjusted at the location from the first condition to a second condition. In the first condition, first and second precursor materials coupled to a graft wall of the graft assembly are substantially respectively isolated from each other and the graft wall is substantially pliable and conformable to the shaped surface. In the second condition, the first and second precursor materials coupled to the graft wall are substantially combined and reacted to form a polymer matrix such that the polymerized graft wall is substantially less pliable than the first condition and further such that the polymerized graft assembly provides a therapeutic result to the tissue structure.

Another aspect of the invention is a method for treating an aneurysm in a patient that includes delivering a graft assembly to a shaped surface of the tissue wall structure when the graft assembly is in a first condition, and then adjusting the graft assembly at the location from the first condition to a second condition. In the first condition, first and second precursor materials coupled to a graft wall of the graft assembly are substantially respectively isolated from each other and the graft wall is substantially pliable and conformable to the shaped surface. In the second condition, the first and second precursor materials coupled to the graft wall are substantially combined and reacted to form a polymer matrix that the polymerized graft wall is substantially less pliable in the second condition than in the first condition and substantially retains the shape of the shaped aneurysm surface as a memory condition for the in-situ polymerized graft wall. A therapeutic result is provided to the aneurysm with the polymerized graft assembly in the second condition.

According to one mode of the foregoing graft assembly aspects, the first and second precursor materials comprise fibrinogen and thrombin, respectively. Fibrin glue is formed in the graft assembly by mixing the fibrinogen and thrombin in the second condition.

According to another mode, the first and second precursor materials are housed within first and second pluralities of reservoirs that are respectively isolated from each other by an erodable material in the first condition. The graft assembly is adjusted to the second condition at the location by eroding the erodable material between the first and second respective pluralities of reservoirs to thereby allow mixing of the first and second precursor materials.

Another aspect of the invention is a method for preparing a custom therapeutic graft that is adapted for use in treating a damaged tissue wall structure within a body of a patient as follows. A sheet of substantially pliable graft material is provided in a first condition that is substantially pliable and that comprises a graft wall and first and second precursor materials coupled to the graft wall and that are respectively isolated from each other. A geometry of the custom therapeutic graft is chosen based upon an anatomical geometry of the damaged tissue wall structure. The chosen geometry is cut from the provided sheet of graft material in the first condition to thereby form the custom therapeutic graft in the first condition. The custom therapeutic graft in the first condition is adapted to be delivered to and conformed to the shape and geometry of the damaged tissue wall structure. The custom therapeutic graft that is formed is also characterized as being convertible from the first condition to a second condition wherein the first and second precursor materials are mixed within the graft wall and polymerize to a substantially less pliable form than the first condition and in a polymerized memory condition substantially conformed to the shape of the damaged tissue wall structure.

According to one further mode of the foregoing method aspects and modes, an external support scaffold is provided to a AAA with an external support scaffold agent sufficient to substantially reduce dilation or progression of the AAA.

According to one embodiment of this mode, the external support scaffold agent is transperitoneally delivered to the location along the AAA through an anterior abdominal access site.

According to another embodiment, the external support scaffold agent is delivered to the location along a AAA from a posterior access site on the patient's back.

In another embodiment, the external support scaffold agent is delivered to the location along the AAA from an axial access site on the patient's side.

According to another further related mode, an external support scaffold is provided to a thoracic aortic aneurysm sufficient to substantially reduce dilation or progression of the thoracic aortic aneurysm.

According to one embodiment of this mode, the external support scaffold agent is transthoracically delivered to the location along the thoracic aortic aneurysm.

In still another further related mode, an external support scaffold is provided to a cerebral aneurysm sufficient to substantially reduce dilation or progression of the cerebral aneurysm.

According to another aspect of the invention, a AAA is treated with an external support scaffold, wherein the AAA being treated is less than about 5.5 or 5.0 centimeters in dilated diameter, and in a further embodiment is less than about 4 centimeters in diameter, and in still a further embodiment less than about 3 centimeters in diameter. According to this benefit, the external support scaffold approach, using minimally invasive laparoscopic techniques in particular, is able to appropriately treat such early detected weakened walls, and at early stages of progression before catastrophic risks are elevated, and without substantially mitigating adverse effects found typically with endograft techniques that are often avoided for such aneurysms at earlier, less dilated stages of progression.

In further related aspects, such size considerations furthermore relate to therapeutic tools and methods for treating other forms of aneurysm at earlier, less dilated geometries and stages, such as in particular thoracic aortic aneurysms, and in some cases cerebral aneurysms.

It is to be appreciated that each of the aspects, modes, embodiments, variations, and features herein described is considered independently valuable and beneficial without requiring combination with the others.

However, notwithstanding the foregoing, their various combinations are also considered to provide additional independent value and benefit, as would be contemplated by one of ordinary skill, and thus such combinations are considered additional independent aspects hereunder.

Further aspects of the invention will be brought out in the following portions of the specification, wherein the detailed description is for the purpose of fully disclosing preferred embodiments of the invention without placing limitations thereon.

### BRIEF DESCRIPTION OF THE SEVERAL VIEWS OF THE DRAWING(S)

The invention will be more fully understood by reference to the following drawing which is for illustrative purposes only:

FIG. 1 shows an anterior view of one external AAA stent-graft embodiment of the invention in one mode of delivery and use related to an AAA in a patient.

FIG. 2 shows a similar anterior top view as FIG. 1, with the stent-graft embodiment shown in a second mode of use in relation to the AAA.

FIGS. 3A-D show partially cross-sectioned end views of the stent-graft embodiment shown in FIGS. 1-2 during various serial modes of use in reference to the AAA and variously in reference to other adjacent anatomical structures in the patient.

FIG. 4 shows an angular perspective view of a portion of another external AAA stent-graft embodiment of the invention.

FIG. 5 shows a side view of one component of the external AAA stent-graft embodiment shown in FIGS. 1-3D.

FIG. 6A shows a transversely cross-sectioned view taken along lines 6A-6A of FIG. 5, and further includes certain additional components of the stent-graft embodiment during one mode of operation.

FIG. 6B shows a similar side view as FIG. 6A, except showing the additional components in another mode of operation, and in context of further components shown related to the external AAA stent-graft embodiment.

FIG. 7A shows a longitudinally cross-sectioned view taken along lines 7A-7A in FIG. 5, and further shows certain additional components of the stent-graft embodiment during a similar mode of operation shown in FIG. 6A.

FIG. 7B shows a similar longitudinally cross-sectioned view as that shown in FIG. 7A, except showing the additional components in another mode of operation similar to that shown in FIG. 6B.

FIG. 8 shows one further component of an external AAA stent-graft assembly adapted for use according to the embodiments shown variously among FIGS. 1-7B.

FIG. 9 shows another component of an external AAA stent-graft assembly adapted for use with the component in FIG. 8 and further according to the embodiments shown among FIGS. 1-7B.

FIG. 10 shows a partially exploded view of certain detail of the arrangement between the components shown in FIGS. 8 and 9 according to use as an external AAA stent-graft assembly.

FIG. 11 shows another external AAA stent-graft embodiment of the invention according to one mode of use in relation to an AAA in a patient.

FIG. 12 shows a top plan view of one introducer sheath embodiment that is adapted to deliver the external AAA stent-graft assemblies according to the various embodiments of FIGS. 1-11 in a minimally invasive, port-access delivery approach according to further embodiments of the invention.

FIG. 13 shows a transversely cross-sectioned view taken along lines 13-13 of FIG. 12.

FIG. 14 shows a similar transversely cross-sectioned side view as that shown in FIG. 13, but according to another embodiment for the introducer sheath.

FIG. 15 shows a side view of one mode of operating a transthoracic introducer sheath for delivering an external AAA support scaffold into a patient's body and further shows certain reference anatomical structures.

FIG. 16 shows an angular perspective view of another external AAA support scaffold embodiment according to the invention.

FIG. 17 shows a transversely cross-sectioned, angular perspective view of the embodiment shown in FIG. 16 during one mode of use in relation to an AAA.

FIG. 18 shows further detail of certain schematic arrangements of various components within the support scaffold shown in FIGS. 16-17 during sequential modes A-D of operation in treating an AAA.

FIGS. 19A-B show various transversely cross-sectioned views of respective schematically represented aspects of another AAA external support scaffold embodiment of the invention.

FIGS. 20A-B show various transversely cross-sectioned views of respective schematically represented aspects of another AAA external support scaffold embodiment of the invention.

FIG. 21 shows a transversely cross-sectioned schematic view of another AAA external support scaffold embodiment of the invention.

FIGS. 22A-C show partial top views of further external support structures suitable for use according to further AAA external support scaffold embodiments of the invention.

FIGS. 23A-C show variously transversely cross-sectioned views of respective schematically represented aspects of additional AAA external support scaffold embodiments of the invention.

FIGS. 24A-B show a top view, and transversely cross-sectioned end view, respectively, of a schematic representation of another AAA external support scaffold embodiment of the invention.

FIGS. 25A-B show a top view, and transversely cross-sectioned end view, respectively, of a schematic representation of still another AAA external support scaffold embodiment of the invention.

FIG. 26 shows a transversely cross-sectioned view of another AAA external support scaffold embodiment of the invention.

FIGS. 27A-B show partial, longitudinally cross-sectioned side views of respective schematically represented features related to various modes of use according to the AAA external support scaffold embodiment illustrated in FIG. 26.

FIG. 28A shows a transversely cross-sectioned end view of a schematic representation of another AAA external support scaffold embodiment of the invention.

FIG. 28B shows a transversely cross-sectioned end view of a schematic representation of another AAA external support scaffold embodiment of the invention.

### DETAILED DESCRIPTION OF THE INVENTION

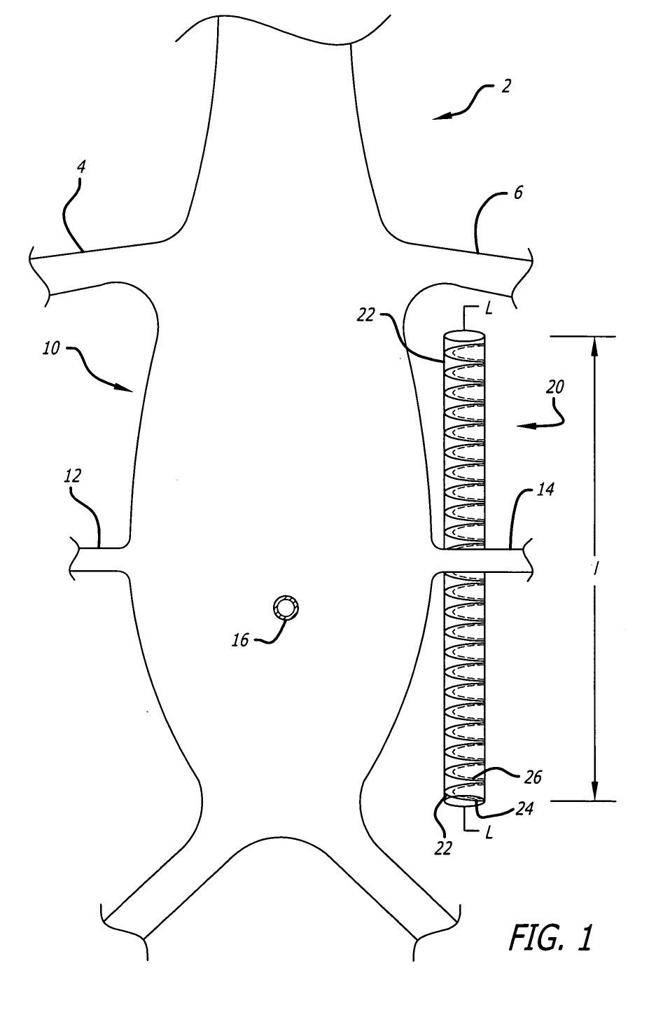

FIG. 1 shows a first embodiment according to the present invention in context of an abdominal aortic vascular tree 2. Tree 2 includes first and second renal arteries 4,6, respectively, an abdominal aortic aneurysm (“AAA”) 10, and various side branch or “perforator” vessels 12,14,16, respectively, extending from the aorta 2 along the AAA 10. An external AAA stent-graft assembly 20 is shown in an anterior view (e.g. looking down onto the exposed aorta from front-to-back, such that the anterior side of the aorta 2 faces out from the page, and the posterior side of the aorta is behind the page).

Assembly 20 includes a plurality of transverse members or fingers 22 located in a linear array along a length I relative to a longitudinal axis L. Each transverse finger 22 includes a graft body 24 with a support scaffold 26 shown in shadow and coupled to graft body 24. FIG. 1 shows the assembly 20 in one mode of use positioned along side of AAA 10 prior to deployment of the external stent-graft assembly 20, and as such stent graft assembly 20 is in a coiled configuration, as will be explained in finer detail below.

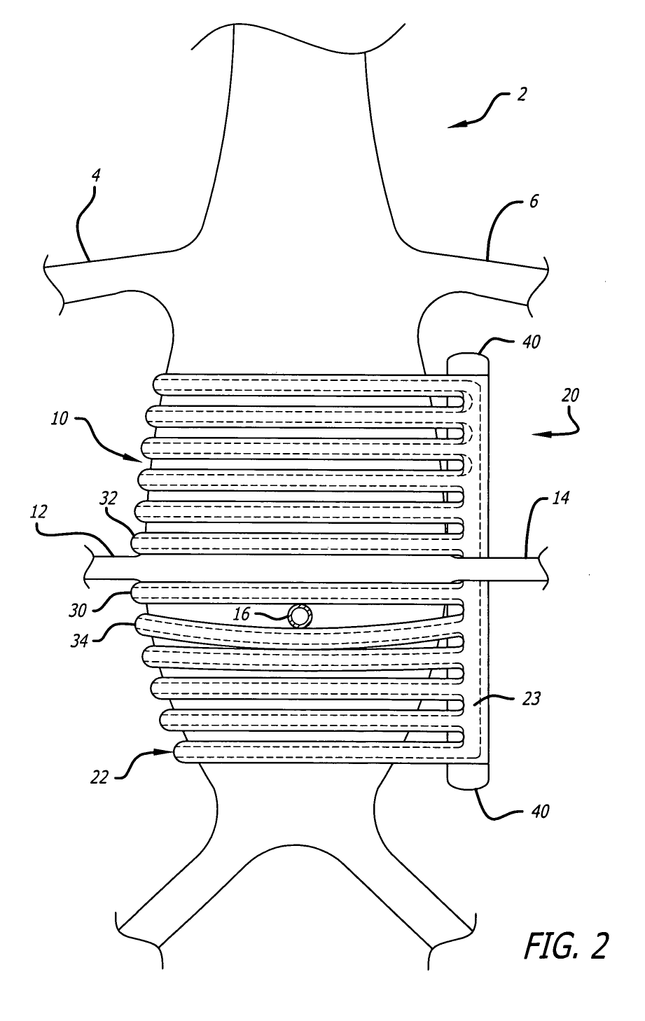

FIG. 2 shows another mode of use, with the array of fingers 22 unrolled to a nearly completely deployed configuration that extends over and around the AAA 10. As shown, the plurality of fingers allow for the assembly to wrap as a substantial cover around the AAA 10 while accommodating the various perforator vessels 12,14,16 between adjacent fingers, as shown at fingers 30,32,34, respectively. The fingers are shaped so as to provide a relatively self-guided arrangement, such that when a side-branch vessel is encountered as the assembly is unrolled up and over AAA 10, the fingers slightly separate and the respective perforator becomes located at a space therebetween.

Whereas this leaves some areas un-supported directly along the AAA 10, e.g. between fingers 30 and 34 where perforator 16 is located, the adjacent fingers nevertheless generally provide sufficient support to improve the overall area, including in the space between these fingers that function as “ribs” for external scaffolding. As further shown in FIG. 2, a delivery member 40 remains coupled to stent-graft assembly 20 along a base 23 thereof where the array of adjacent fingers 22 are integrated together, such as a spine.

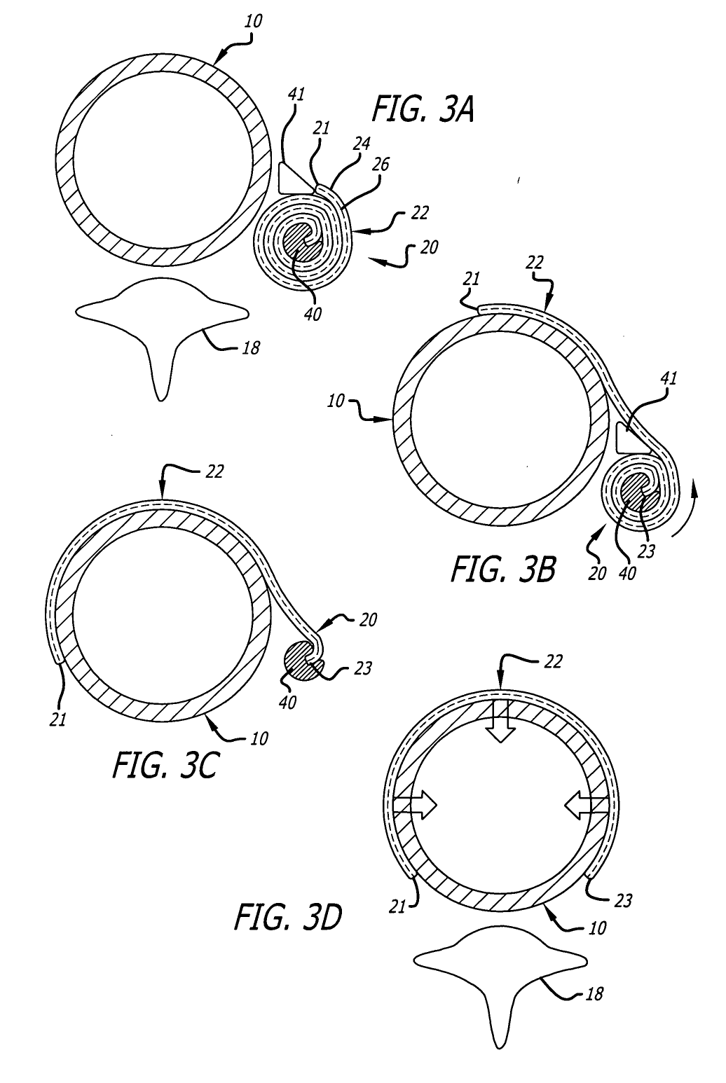

Further detail of the serial modes of operation to adjust the assembly 20 from the first configuration of FIG. 1 to the second substantially deployed configuration of FIG. 2 are further provided by reference to the various side views of FIGS. 3A-D as follows.

FIG. 3A shows assembly 20 in one mode of operation corresponding to that shown in FIG. 1 in the context of a AAA 10 and adjacent spinal body 18 for anatomical context purposes. Assembly 20 includes a graft body 24 coupled to a support scaffold 26 and that is wrapped around a delivery member 40. Delivery member 40 is torqueable, and when torqued can unwind assembly 20, such as over a stand-off 41 that may be for example a deployable mandrel, balloon, etc. In the embodiment shown, stand-off 41 is shaped to provide a ramp over which end portion 21 of stent-graft assembly 20 may traverse to unwind from delivery member 40 and over AAA 10.

As shown in FIG. 3B, delivery member 40 is turned so as to unwind end 21 of assembly 20 up and over the anterior aspect of AAA 10. A further progression is shown in FIG. 3C near completion, but with end 23 of assembly 20 still coupled to delivery member 40. A final result is shown in FIG. 3D, whereas assembly 20 is deployed around the majority of AAA 10, as shown along the anterior circumferential aspect between ends 21 and 23. Though this is not completely around AAA 10, it is considered a substantial improvement to assist against any progression or harmful effects of AAA 10, as the most posterior aspects left unsupported by assembly 20 between ends 21 and 23 are generally better supported by structures along spine 18.

Moreover, the assembly 20 has a memory to the contracted, wound configuration, and as such applies a certain degree of support force inward to AAA 10, and in particular resists dilating forces, such as during peak pressures of systole, etc., which may otherwise create further damage, rupture, or progression of AAA without this support.

It is to be appreciated that the memory condition for assembly 20 may be to the wound configuration, or to another resulting shape of curvature sufficient to provide the intended external support scaffold to a radially enclosed vessel as herein further developed. In the latter case, assembly 20 is held in an elastically deformed configuration in the wound shape until released therefrom that shape and deployed in-situ.

In addition, further embodiments (not shown) however contemplate traversing the stent-graft assembly completely around AAA 10, such as two opposing end portions 21 and 23 coming back together, where they may remain disengaged, or may be coupled together, such as via adhesive or sewing, stapling, stitching, etc.

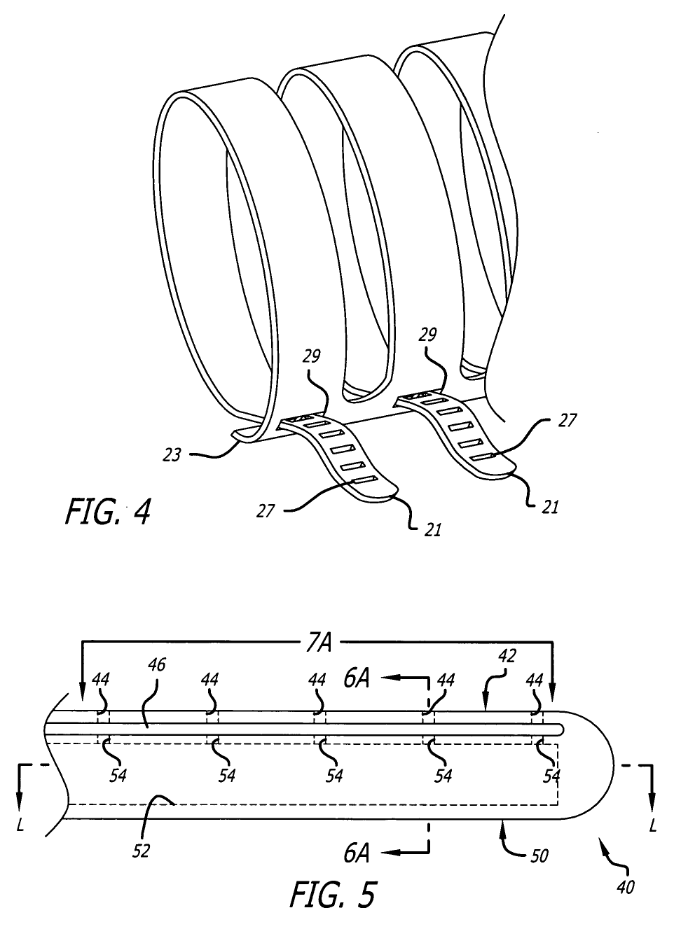

Or, as shown in FIG. 4, ratcheted apertures 29 located at one end 23 may receive protrusions or fingers from the other end 21 that further include a linear array of apertures 27 that engage a protrusion within apertures 29 in a ratchet lock mechanism. After achieving the desired circumference to meet the particular needs of an AAA being treated, remaining excess portions of end 21 may be cut off, such as via a surgical cutting tool, or remain in the body as shown.

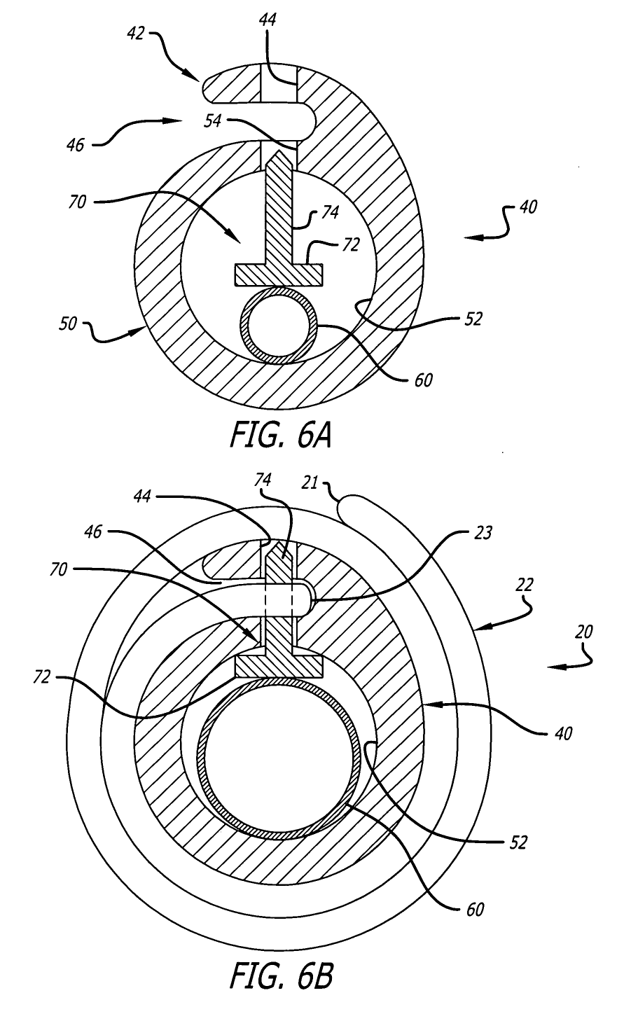

Further detailed embodiments of various components for deployment are provided as follows by various reference to FIGS. 5-6B.

FIG. 5 shows various finer detail aspects of delivery member 40, which includes a housing with a first portion 42 and a second portion 50 that are positioned with a groove 46 formed therebetween. First portion 42 includes a longitudinal array of spaced transverse passageways 44 that communicate into groove 46. Second portion 50 includes a main passageway 52 that communicates externally into groove 46 via a second longitudinally spaced array of transverse passageways 54. Each of transverse passageways 44 is registered with one of transverse passageways 54.

FIG. 6A shows further detail in a cross-sectioned view of the first and second portions 42,50, groove 46, and transverse passageways 44,54, in further context of additional operational components located within main passageway 52 as follows. In one regard, main passageway 52 houses a needle assembly 70 that includes a pointed shank 74 extending radially from a base 72. Also housed within main passageway 52 is an actuator 60 that is an inflatable member in the embodiment shown, and in a deflated condition in the mode shown in FIG. 6A. In the disengaged configuration shown in FIG. 6A, inflatable member 60 is deflated such that needle assembly 74 is withdrawn principally into main passageway 52.

As shown in FIG. 6B in an engaged configuration, inflatable member 60 is inflated and pushes needle assembly 70 via engagement with base 72 laterally within main passageway 52, such that pointed shank 74 extends through transverse passageway 54, across groove 46, and into transverse passageway 44. External AAA stent-graft 20 is further shown in the wound configuration coupled to delivery member 40 in order to illustrate the inter-cooperation between those components. Needle shank 74 pierces through end portion 23 of stent-graft 20 within groove 46, thus coupling delivery member 40 with stent-graft 20. Accordingly, torsional rotation of the delivery member 40 in the counterclockwise direction unwinds stent graft 20, whereas torsional rotation in the clockwise direction may re-wrap stent-graft 20. The latter may be performed, for example, if deployment on a first effort did not produce desirable results, such that the stent-graft can be re-wound, repositioned, and tried again, or such that the assembly removed so as to abort the procedure, or make a modification to the first chosen device, or to another subsequent chosen device.

By deflating inflatable member 60, a biased recovery of needle assembly 70 back to the disengaged configuration shown in FIG. 6A releases the coupling between delivery member 40 and stent-graft member 20 via needle assembly 70. This allows the stent-graft member 20 to be released in the deployed configuration as an external AAA stent-graft implant.

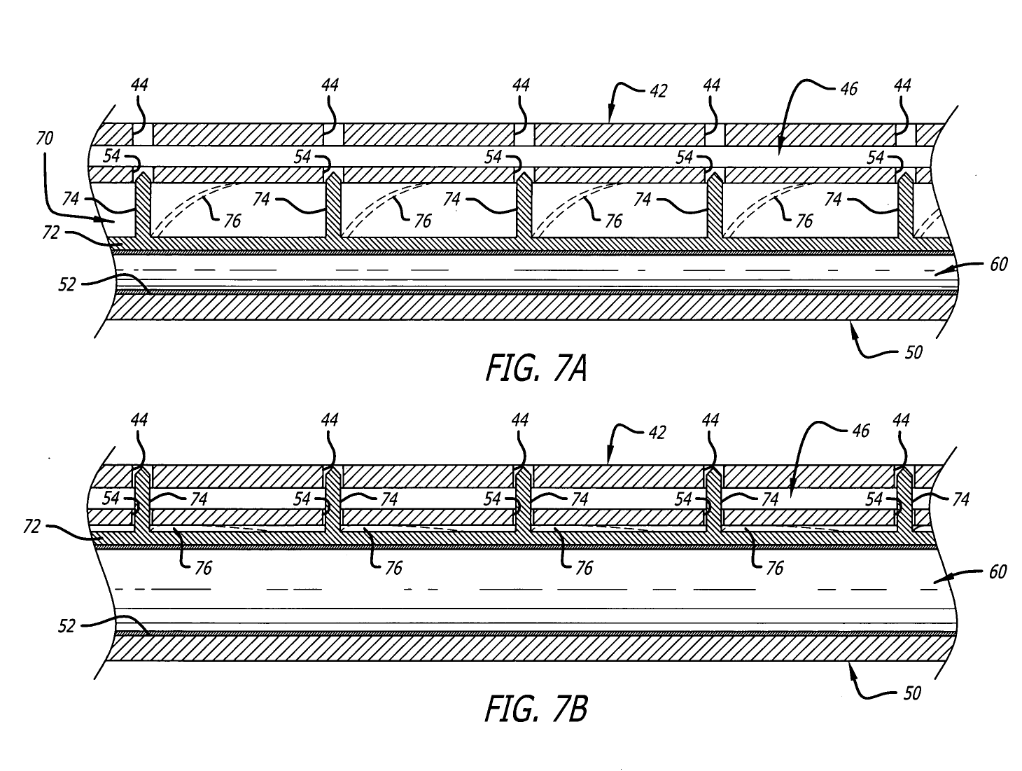

According to the longitudinally cross-sectioned side view of delivery device 40 shown in FIG. 7A, in that particular illustrative embodiment needle assembly 70 actually includes a base 72 that extends between and coupled to a linear array of needle shanks 74, each registered with and adapted to be advanced through apertures 54 and 44 of portions 50 and 42, respectively, and across groove 46. Accordingly, one actuated mechanism allows for engagement between delivery member 40 and a stent graft (not shown in FIGS. 7A-B for clarity) along a length desired for controllability from one end of the graft to the other.

As shown in the transverse side view for FIGS. 6A-B, FIGS. 7A-B show in a longitudinally cross-sectioned perspective the respective components of delivery member 40 in disengaged and engaged configurations, respectively, corresponding with deflated and inflated conditions for inflation member 60, also respectively. As further shown in these longitudinal side views, however, a bias system is provided with an array of leaf springs 76 corresponding with needle shanks 74 that force the shanks 74 aside within main passageway 52 when inflation member 60 is deflated, thus pulling the shanks 74 to the disengaged condition outside of groove 46 to thus release a stent-graft end there (or receive one for later engagement). These leaf springs in the embodiment shown are secured at one end to base 72 of needle assembly 70, and are adapted to compress against a memory bias under an applied force from the inflation of inflation member 60, as shown in FIG. 7B.

Subsequent deflation of inflatable member 60 allows springs 76 to recover with a recovery force that pulls shanks 74 back down the respective apertures and out from groove 46.

Further details regarding the stent-graft assembly for use according to the prior embodiments are provided for further illustration as follows.

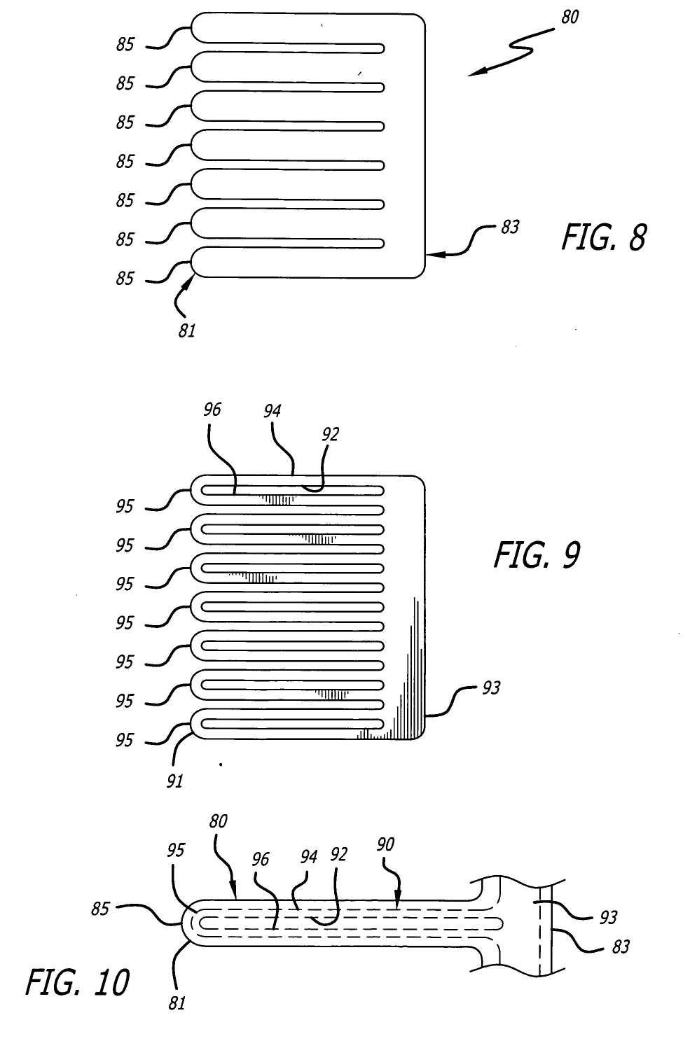

FIG. 8 shows a portion of a graft material 80 that is for example a sheet cut into a pattern that includes a spine portion 83 along one end, with a plurality of fingers 85 extending therefrom to an opposite end 81. Graft material 80 may be for example polytetrafluoroethylene (PTFE), or other suitable graft material as would be apparent to one of ordinary skill.

FIG. 9 shows a portion of a support scaffold 90 that is cut for example from a sheet into a pattern with a spine portion 93 along one end, with a plurality of fingers 95 extending therefrom to an opposite end 91. Each finger 95 is cut with a hollow central portion 92 with two lateral arms 94,96 in the particular embodiment shown. This provides more flexibility as a support scaffold, and may be modified in terms of dimensions to meet a particular application or need. Support scaffold in one highly beneficial embodiment is constructed from a super-elastic material, such as an alloy, and in particular beneficial embodiments is nickel-titanium alloy. Such may be cut for example from a sheet of material, e.g. with a laser.

Support scaffold 90 is coupled to graft material 80, as shown for further illustration in FIG. 10, which may be accomplished in many ways. In one further embodiment not shown, scaffold 90 may be sandwiched between two parallel panels of graft material 80, which may be thereafter sealed around scaffold 90, such as by induction or laser welding of the like materials together of the opposite graft sheets, or such as for example with adhesive bonding or other form of heat or solvent bonding. In another embodiment also not shown, scaffold 90 may be put between two parallel sheets of graft material that are not at that point cut into patterns. Such pattern may be thereafter cut around the scaffold 90, such as by use of a laser, either before, after, or during welding the graft sheets together in the appropriate pattern to seal around the patterned scaffold 90.

In still further embodiments not shown, the support scaffold 90 may be stitched to the mating graft material 80, either on one side of a single graft sheet, or between two sheets. Still further, apertures formed within graft material 80 may be formed, through which the fingers 95 of support scaffold 90 traverse from one side to the other of the graft material 80, resulting in an overall coupling such that support scaffold 90 carries graft material 80 with it through various shape configurations during use and deployment. Each of these are considered beneficial examples that are illustrative of the breadth by which many different arrangements may be accomplished to achieve the intended purpose without departing from the intended scope hereof.

The particular geometries and respective arrangements of support scaffold and graft materials to form a longitudinal array of patterned fingers, though highly beneficial and providing specific beneficial uses, is illustrative of broad aspects of the invention that provide external support scaffold around the exterior of a AAA. Moreover, providing such that is capable of delivery and deployment transthoracically and with minimal incision and invasion trauma is of further substantial benefit. However, other arrangements and configurations to achieve such are also contemplated, whether by other forms of stent-graft composite members or otherwise.

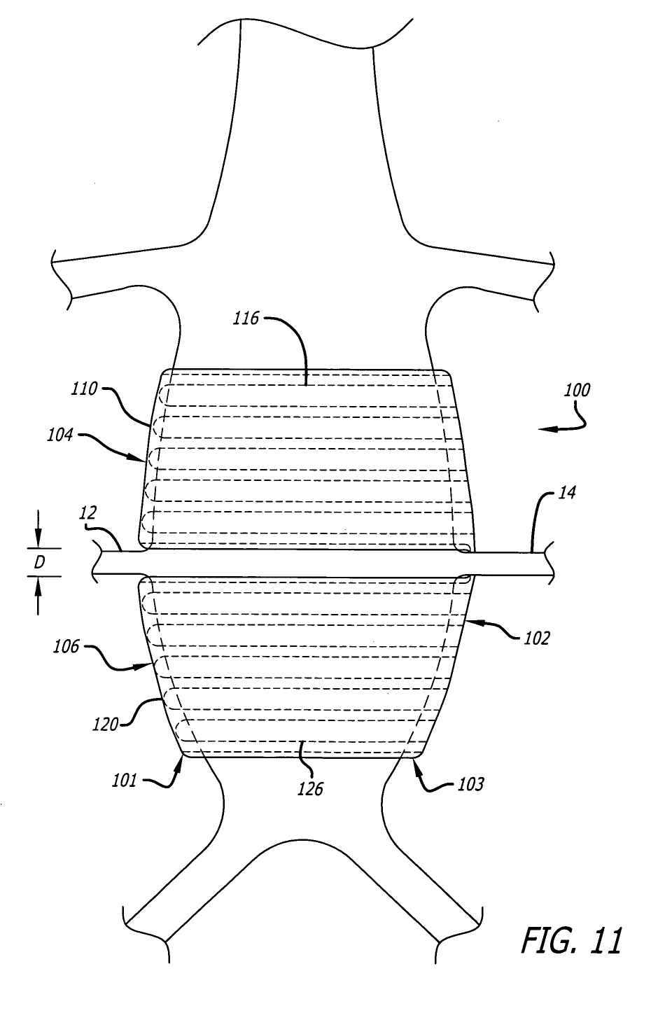

For example, FIG. 11 shows another external AAA stent-graft system 100 with two discrete portions 104,106, respectively, that extend around AAA from one transverse end 103 to the other transverse end 101, and are spaced by a distance D along longitudinal axis of the respectively engaged AAA. This embodiment otherwise may be delivered and deployed in a similar manner and with similar delivery device mechanisms as previously disclosed above.

These discrete portions 104,106 include support scaffolds 116,126, respectively, that include longitudinally spaced fingers as previously described for the previous embodiment. However, these support scaffold fingers are coupled within more uniform graft portions 110, 120, also respectively, that do not include longitudinally spaced fingers or gaps. The result is more uniform across the AAA 10, and is adapted for use where implantation corresponds with known anatomy such that the more discrete separation D corresponds with particular locations of perforator vessels such as at 12 and 14, respectively.

This arrangement may provide, for example, for more extensive and uniform support along the various regions of the AAA, but may be limited to use along only predetermined portions of a AAA, or again at regions with known perforator anatomy corresponding with the spaces provided between graft portions.

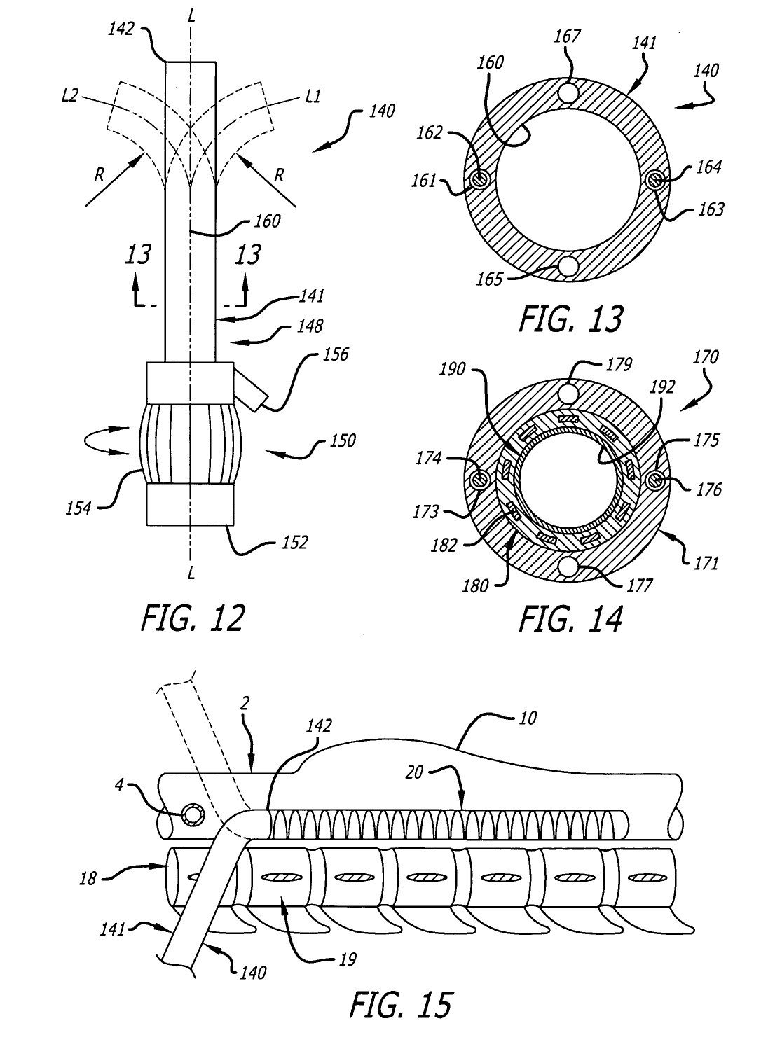

Various modes of delivering the systems described by reference to the prior embodiments above are also contemplated. However, one particular beneficial embodiment is herein shown in FIGS. 12-15 and described as follows. FIG. 12 shows an introducer sheath 140 that has an elongate body 141 with a deflectable tip 142 that is deflectable between a relatively straight longitudinal axis L and in either of two directions with a radius R along right or left deflections corresponding with longitudinal axis L1 or L2, respectively. A proximal end portion 148 includes a control handle 150 with a coupler 152 that may be for example a hemostatic valve that provides for sealed introduction of devices therethrough and into a main lumen 160 (shown in shadow). Handle 150 further includes an actuator assembly 154, which in the embodiment shown actuates tip deflection upon rotating the actuator manually externally of the patient, as indicated by double-headed reference arrows.

Further couplers may be provided into additional lumens, as may be required for a particular arrangement, as shown at coupler 156. Such may be used for example for infusion of fluids, such as liquids or gas, for example in order to pressurize the region externally from tip 142 to deflect structures from around the device being deployed therethrough. Moreover, other lumens may be provided that house for example fiber optic light sources and/or imaging fibers, laparoscopic surgical instruments for resection, stitching, etc., or combinations of such exemplary additional adjunctive uses. Such examples are considered as useful aids to the proper placement and deployment of the various external scaffold embodiments herein shown and/or described according to further overall system embodiments herein contemplated.

One particular cross-section through elongate member 140 is shown in FIG. 13, and includes a main lumen 160 surrounded by a plurality of satellite lumens 161,163,165,167 separated by 90 degrees, respectively. Pull wires 162,164 are located within opposite lumens 161,163, respectively, for a bi-directional tip control. Satellite lumens 165,167 are shown empty, but may house various devices, components, or materials (e.g. fluids, gas, etc.) as would be apparent to one of ordinary skill upon review of this disclosure and by further reference to other available related information. Or, they may house further pull-wires for three or four plane tip control. Nonetheless, by providing bi-directional control in combination with torqueability of the overall structure, such should provide sufficient degrees of freedom in all directions to provide the necessary orientation for tip 142 to deliver the external AAA scaffold device assembly as desired.

Further modifications are contemplated, as shown for further illustration in FIG. 14 that shows a shaft 170 with an outer tubular layer 171 that includes lumens 173,175,177,179 and two opposite pull-wires 174,176 in a similar arrangement to the overall prior embodiment. However, in the present embodiment, a more composite structure is provided with an intermediate layer 180 that is a ribbon-reinforced (e.g. braided or wound) composite member with ribbon 182 that provides radial support to maintain roundness during deflection or bending. An inner layer 190 provides a lubricious surface, such as for example polytetrafluoroethylene (PTFE) or fluoroethylene polymer (FEP). Such multi-layered composite construction may be accomplished according to a variety of methods known to one of ordinary skill in the art, but for illustration may incorporate heat bonding, assistance of outer heat shrink tubings to “bundle” the concentric layers under radial force, adhesives, solvent bonding, etc.

FIG. 15 shows one example of a delivery technique using introducer sheath 140 to deliver the desired external AAA scaffold system 20 to the desired location along an AAA 10 for deployment over and around AAA 10 as a support scaffold implant, as shown in one exemplary embodiment and mode of use in FIG. 15. More specifically, FIG. 15 shows a dorsal or posterior approach from the patient's back, introducing introducer sheath 140 through intercostals spaces between adjacent ribs (or transverse spinal processes where introduced below the rib cage) 18,19 along the patient's back and at a location corresponding with aorta 2 that is inferior to renal artery 4 but superior to the beginning of AAA 10. With the shape provided at tip 142 as shown, a substantially inferior delivery orientation is provided. External AAA scaffold assembly 20 is thus delivered therethrough along side of aorta 2 and across the length of AAA 10. Subsequent deployment and detachment as an implant, as described above, completes the procedure (with of course the additional use of necessary tools and procedures for tissue resection or dissection, e.g. connective tissues, pleurae, etc, and appropriate wound closure following implantation). Further techniques are also contemplated, such as for example a ventral or anterior approach from the anterior side of aorta 2, as shown for illustration in shadow (e.g. transperitoneal approach).

Or, in a further example, the various introducer and related implant assemblies may be delivered from a more inferior position with a superior orientation from the tip 142 of the respective introducer sheath 140.

The foregoing embodiments provide examples of highly beneficial stent-graft embodiments, and related delivery embodiments, for providing transthoracic, external AAA support scaffolds. However, other embodiments are also contemplated that achieve external AAA support scaffolds that do not employ stent-grafts as the scaffold.

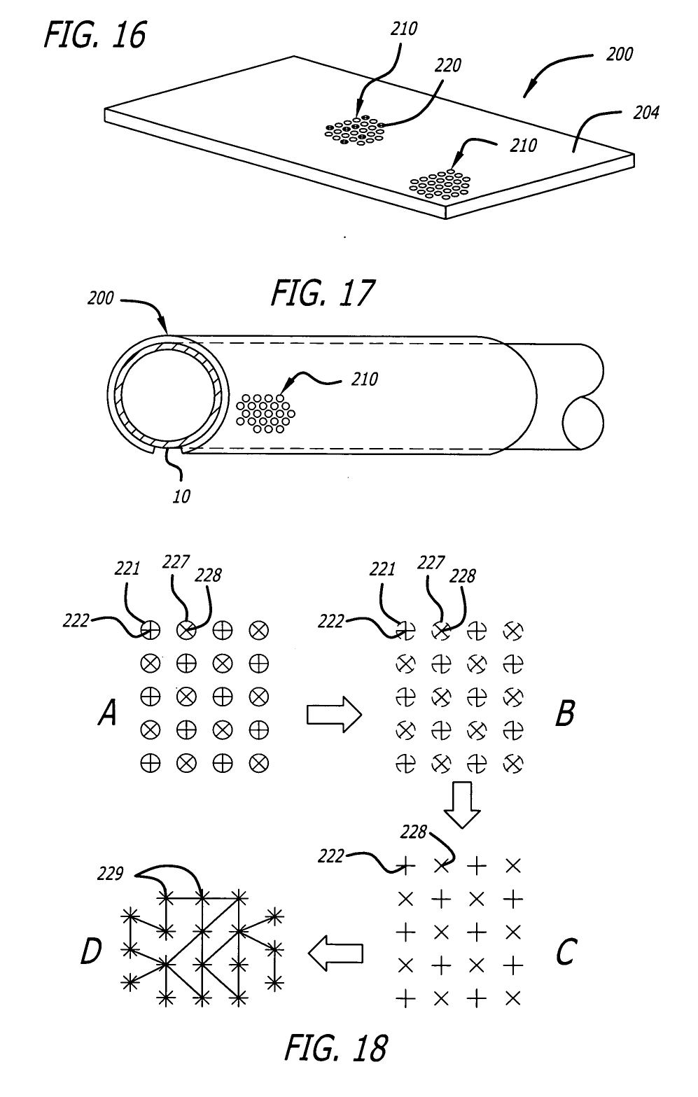

For example, FIG. 16 shows a plan view illustration of a novel type of graft sheet 200 that is constructed to provide an external AAA support scaffold in the following manner. Graft sheet 200 includes a sheet material 204 with a plurality of pockets or reservoirs 210 that house one or more materials 220 that, in a first condition, results in graft sheet 200 being substantially pliable.

In this condition, the graft sheet 200 may be wrapped around the external surface of a AAA 10, as shown in FIG. 17. However, once so arranged, the graft material is treated in a manner that converts the material 220 into a more rigid support material, essentially shaping the graft sheet 200 into the memory shape it is in upon such conversion.

This may be accomplished in one beneficial embodiment by providing material 220 as a light sensitive material that polymerizes upon exposure to certain types of light, such as UV curable adhesives that are appropriately biocompatible according to the particular application within the sheet 200 (e.g. may be completely stored within an outer protective layer, in which case biocompatibility concerns may be reduced).

However, in a particular beneficial embodiment illustrated in the embodiment of FIG. 18, material 220 is actually two pre-cursor materials, 222, 228 that are stored within two distinct arrays of pockets 221,227, respectively, within sheet material 204. These two pre-cursor materials, when combined, provide a reaction that provides a hardened material, such as a polymerization resulting in a supportive polymer matrix. In one highly beneficial mode, the two pre-cursor materials are fibrinogen and thrombin, which when mixed form fibrin glue, a biopolymer with substantial beneficial results.

Such mixing of precursor materials may be accomplished for example by providing the sheet material 204 as a bioerodable or biodegradable material, such that upon the decay of the scaffold that separates the pre-cursors, they mix to polymerize the matrix. This is shown in progressive modes for example between conditions designates as “A”, “B”, “C”, and “D” in FIG. 18 yielding ultimately the fibrin glue 219 as an external support scaffold surrounding the exterior of a AAA.

Another benefit of such a novel graft sheet 200 is that it may be cut and arranged along the desired region where it is to “set up” as a support scaffold, and left to adjust automatically to the support condition in that shape and configuration. For example, portions may be cut out where perforators are known to extend from a particular AAA, customizing the external graft sheet in the pliable condition that later sets up as a support scaffold in that customized shape. Moreover, such cuts may be made and after placement of a perforator between two separated graft pieces on either side of the cut, these sides may be stitched together on either side of the perforator to provide more complete coverage of all areas of the AAA.

Other modifications are contemplated. For example, a sheet material may be provided impregnated with only one of two-precursor materials. The second precursor material may be applied to the graft material when it is in the desired shape around the AAA and polymerization or otherwise “set-up” of the material is desired. Such may be applied for example with a syringe or spray nozzle. For example, either of fibrinogen or thrombin may be impregnated into the graft, whereas the other is applied to the AAA before or after applying the graft around the outside of the AAA in the pliable condition.

Further to this type of embodiment, a variety of graft materials may be employed, and need not be biodegradable or erodable, so long as mixing of the fibrinogen and thrombin, or other multi-part (e.g. two-part) precursor materials, is allowed. Such may be allowed for example with appropriate porosity into the microscopic reservoirs of the impregnated graft.

It is contemplated that the benefits of polymerization of an exterior AAA support member from a pliable condition to a more rigid support condition may be further applied in combination with the prior stent-graft embodiments, or in other stent-graft or other scaffold embodiments herein or otherwise previously contemplated or as presently anticipated in the art (or as otherwise modified in obvious variations therefrom by one of ordinary skill).

Moreover, it is further contemplated that application of fibrin glue as a highly biocompatible biopolymer to the outer surface of a AAA may provide substantial benefit as a support scaffold, even without any type of accompanying graft or other support material. One or more needles for example may be used to spray fibrinogen and thrombin onto the outer surface of a AAA over sufficient area to strengthen the wall and provide support to prevent further progression of the AAA dilation, or prevent degradation such as deadly ruptures. Such may be performed under direct surgery, or using transthoracic approaches such as similar to those herein described or as otherwise apparent to one of ordinary skill based upon a thorough review of this disclosure. For example laparoscopic techniques may be used through abdominal incisions and accompanying tissue resections and/or dissections as appropriate to expose fluid injection devices to at least a substantial portion of the anterior aspect of an AAA. Subsequent fibrin glue delivery to that surface will strengthen it, again at least along a substantial anterior portion where most dilation occurs (and the least surrounding tissue support prevents progression).

In further highly beneficial embodiments, cell therapy may be combined with fibrin glue or other molecular agent delivery. For example, autologous cells may be taken before and cultured, and then delivered in a manner concomitant with fibrin glue delivery. Fibrin glue delivery in combination with certain cell delivery, such as for example autologous myoblasts or fibroblasts, has been demonstrated to provide substantially beneficial tissue scaffolds to cardiac structures, and in fact is believed to prevent progression of, and perhaps stimulate regression of, weakened cardiac structures such as related to CHF or infarct. In this very different application of the present invention, tissue scaffolds are created along an exterior surface of a AAA to provide an outside-in approach to strengthening that otherwise weakened vessel wall.

Such applications of fibrin glue, or tissue therapy, or combinations thereof, may also provide substantial benefit to internal scaffold procedures according to a novel addition to more conventional techniques. More specifically, such a scaffold may be delivered behind an endo-aortic stent-graft, e.g. between the graft and the AAA wall. As such polymerizes and “sets-up,” the area behind the AAA endo-graft is strengthened and adhered to the graft and the wall. Such prevents movement of the graft over a long-term implant life, and also promotes a healthy, solid tissue environment behind the graft.

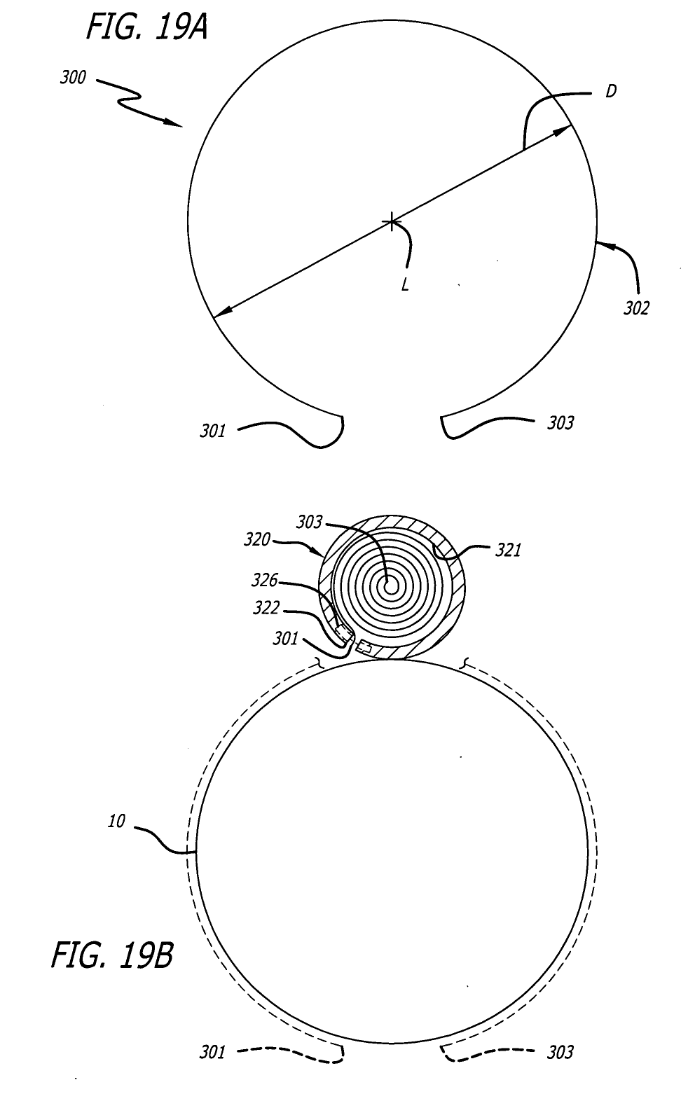

FIGS. 19A-B illustrate a further embodiment of the invention as follows.

As shown generally in FIG. 19A in a deployed configuration, an external AAA support assembly 300 includes an adjustable wall 302 with first and second transversely opposed ends 301,303, respectively. As shown in FIG. 19A, AAA support assembly 300 has a memory condition that at least partially circumscribes a diameter D transverse to a longitudinal axis L, such that ends 301,303 face each other relative to the circumference around axis L. AAA support assembly 300 is adjustable to a first configuration shown in FIG. 19B and that constitutes a deformed configuration from the memory condition shown in FIG. 19A as follows. Assembly 300 is forced into a radially confining lumen 321 of delivery sheath or member 320, and thus is deformed into a deformed shape, which in the particular illustrative embodiment shown in FIG. 19B is in a rolled configuration, also generally referred to as a “cigarette” rolled type of configuration. Sheath 320 also includes a slit 322 through which transverse end 301 is engaged.

An adjustable lock assembly 326 is shown schematically in shadow in FIG. 19B and is adjustable between an engaged configuration and a disengaged configuration. In the engaged configuration, end 301 is held relatively fixed relative to slit 322 such that assembly 300 is held in the first configuration during delivery along aorta 10. In the disengaged configuration, end 301 is released from engagement such that recovery force from assembly 300 within lumen 321 forces end 301 out through slit 322. In continued disengagement of lock assembly 326, support assembly 300 continues to unravel through slit 322 and around AAA 10, as shown in shadow in FIG. 19B.

FIG. 19B shows AAA support assembly 300 and delivery sheath 320 positioned along an anterior aspect of AAA 10 (e.g. the spine, not shown, would be located along an opposite posterior aspect of AAA 10, substantially on the opposite side of delivery sheath 320 and assembly 300 as shown in FIG. 19B). According to the present embodiment and this mode of delivery and use, it is to be appreciated that delivery sheath 320 may require repositioning, either through rotation or further circumferentially around AAA 10, in order to completely release AAA support assembly 300 therefrom such that end 303 is deployed in opposite orientation around AAA 10 than end 301.

Moreover, upon substantially deploying the first end 301, such as shown in shadow in FIG. 19B, the remainder of AAA support assembly 300 not yet unwound may suitably self-deploy to the desired configuration upon simply longitudinally removing delivery sheath 320. In other words, material recovery from the wound deformed configuration to the memory configuration may be sufficient to complete full deployment following partial deployment through a side slit such as slit 322. Or, the delivery assembly may be alternatively given a more axial deployment position accommodating the one sided deployment, such as similar to that previously described by reference to FIGS. 3A-D

The adjustable lock assembly 326 may be adjusted between engaged and disengaged configurations during deployment of AAA support assembly 300, in order to control the deployment in a managed fashion. Many different particular mechanisms may be employed to achieve such adjustable lock, and may be integrated mechanism within the walls of delivery sheath 320 as shown in illustrative schematic shadow, such as deployable and retractable needles for example similar in some regards to the embodiments of FIGS. 5-7B. Or, a rotatable mechanism may be provided that achieves a normal force on the AAA support assembly 300 along slit 322 or adjacent thereto.

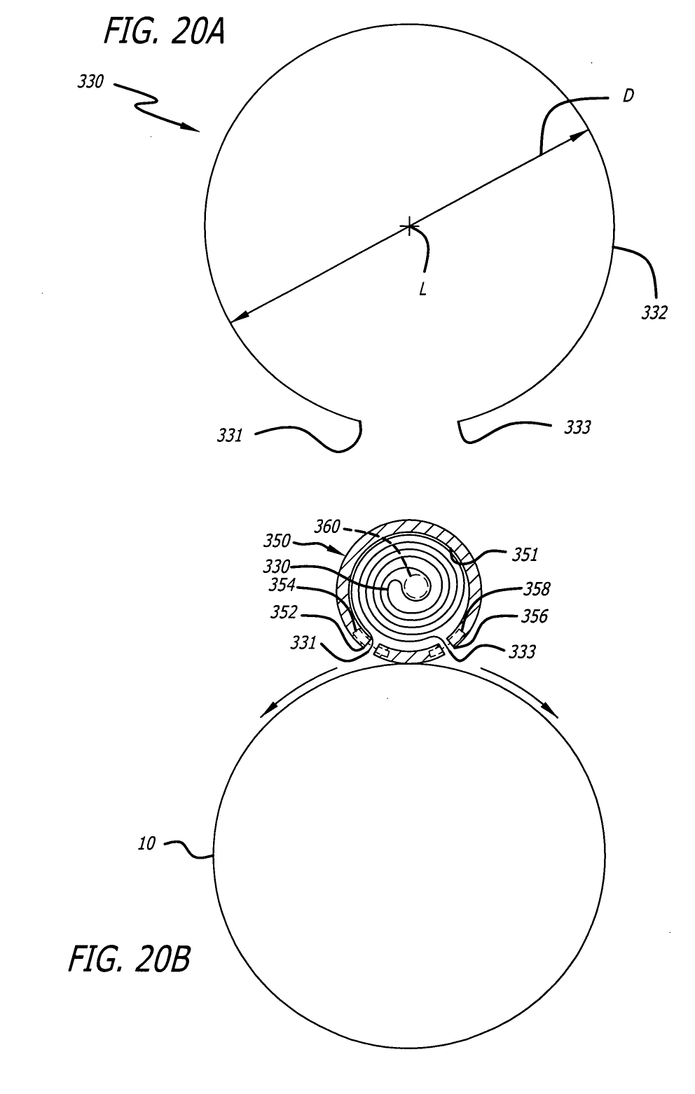

FIGS. 20A-B illustrate a further embodiment of the invention as follows.

As shown generally in FIG. 20A in a deployed configuration, an external AAA support assembly 330 includes an adjustable wall 332 with first and second transversely opposed ends 331,333, respectively. As shown in FIG. 20A, AAA support assembly 330 has a memory condition that at least partially circumscribes a diameter D transverse to a longitudinal axis L, such that ends 331,333 face each other relative to the circumference around axis L.

AAA support assembly 330 is adjustable to a first configuration shown in FIG. 20B and that constitutes a deformed configuration from the memory condition shown in FIG. 20A as follows. Assembly 330 is forced into a radially confining lumen 351 of delivery sheath or member 350, and thus is deformed into a deformed shape, which in the particular illustrative embodiment shown in FIG. 20B is in a rolled configuration, also generally referred to as a “cigarette” rolled type of configuration. Sheath 350 also includes first and second slits 352,356 through which transverse ends 331,333 are engaged, respectively.

First and second adjustable lock assemblies 354,358 are shown schematically in FIG. 20B and are each adjustable between an engaged configuration and a disengaged configuration as follows. In its respective engaged configuration, ends 331,333 are held relatively fixed relative to slits 352,356 such that assembly 330 is held in the first configuration during delivery along AAA 10. In each respective disengaged configuration, ends 331,333 are released from their respective engagement with locks 354,356, respectively, such that memory recovery force from assembly 330 within lumen 351 forces end 331 out of slit 352, and forces end 333 out of slit 356.

In continued disengagement of lock assemblies 354,358, AAA support assembly 330 continues to unravel through slits 352,356 and around AAA 10, as shown by way of opposite pointing vector arrows exiting slits 352,356 in FIG. 20B.

FIG. 20B shows AAA support assembly 330 and delivery sheath 350 positioned along an anterior aspect of AAA 10. According to the present embodiment and this mode of delivery and use, it is to be appreciated that delivery sheath 350 may not generally require substantial transverse repositioning around AAA 10 for substantially complete deployment of AAA support member 330 around AAA 10 as desired. This is due to the bilateral deployment via slits 352,356 for opposite ends 331,333 of AAA support member 330., either through rotation or further circumferentially around AAA 10, in order to completely release AAA support assembly 330 therefrom such that end 333 is deployed in opposite orientation around AAA 10 than end 331.

It is at least in part for this reason that the present bi-laterally deployable embodiment is highly beneficial for an anterior approach for delivery and deployment around a AAA, as shown in FIG. 20B.

Either or both of the adjustable lock assemblies 354,356 may be adjusted between engaged and disengaged configurations during deployment of AAA support assembly 330, in order to control the deployment in a managed fashion. Many different particular mechanisms may be employed to achieve such adjustable lock, and may be integrated mechanism within the walls of delivery sheath 350 as shown in illustrative schematic shadow, such as deployable and retractable needles for example similar in some regards to the embodiments of FIGS. 5-7B.

Or, other mechanisms may be used as would be apparent to one of ordinary skill based upon review of this disclosure and other available information. For example, a rotatable mechanism, e.g. similarly slitted coaxial member (not shown) may be provided for further example within or around sheath 350. Such may be selectively rotated relative to sheath 350 and slit 352 for example in order to achieve either a disengaged keyed relationship between the adjacent slits, or an engaged relationship via a skewed positioning of the respective slits to provide a normal force of engagement on the AAA support assembly 330.

Further beneficial embodiments are to be appreciated as well, such as modifications to the foregoing anteriorly delivered embodiments of FIGS. 19A-20B. For example, other locations may be chosen to achieve the adjustable locking with AAA support assembly 300 or 330 for controlled delivery and deployment thereof. This includes for example via a rotatable internal member, such as shown for illustration at member 360 shown schematically in shadow in FIG. 20B, which may be for example releasably engaged to AAA support assembly 330 within the rolls of assembly 330 within lumen 351. In similar manner, an adjustable rotatable engagement assembly (not shown) may be engaged to opposite end 303 within the rolled support member 300 shown in FIG. 19B, such as in similar manner to that described for other embodiments hereunder (e.g. FIG. 6B).

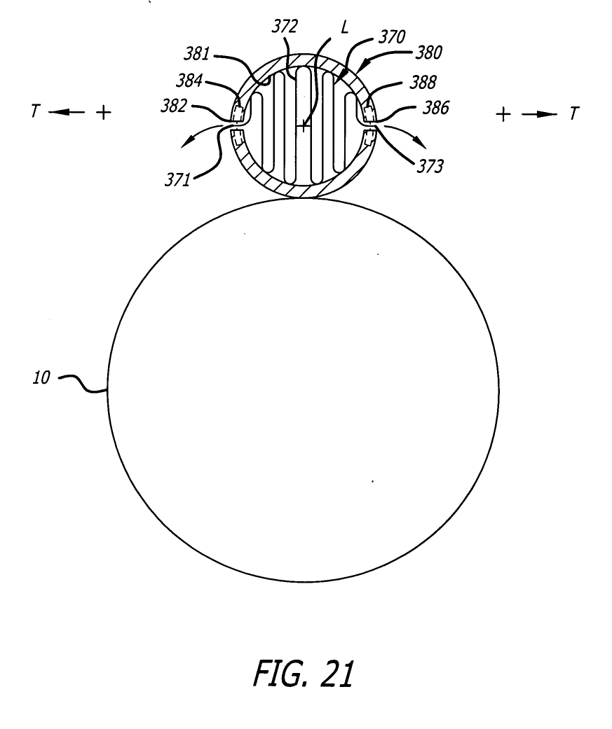

Another anterior delivery approach and assembly is illustrated by way of further example in FIG. 21. FIG. 21 shows a delivery configuration for external AAA support assembly 370 that includes an adjustable wall 372 with first and second transversely opposed ends 371,373, respectively. As shown in similar regards to other embodiments of FIGS. 19A-20B, AAA support assembly 370 has a memory condition that at least partially circumscribes a diameter D transverse to a longitudinal axis L, such that ends 371,373 face each other relative to a circumference around axis L.

AAA support assembly 370 is adjustable from the memory condition and shape to a first configuration shown in FIG. 21 and that constitutes a deformed configuration from the memory condition. Assembly 370 is forced into a radially confining lumen 381 of delivery sheath or member 380, and thus is deformed into a deformed shape, which in the particular illustrative embodiment shown in FIG. 21 is in a transversely “accordioned” configuration, also generally referred to as a “serpentine” folded configuration, that is folded along a transverse axis T that is transverse to a longitudinal axis L. Sheath 380 also includes first and second slits 382,386 through which transverse ends 371,373 are engaged, respectively.

First and second adjustable lock assemblies 384,388 are shown schematically in FIG. 21 and are each adjustable between an engaged configuration and a disengaged configuration as follows. In their respective engaged configurations, ends 371,373 are held relatively fixed relative to slits 382,386 such that assembly 370 is held in the first configuration during delivery along AAA 10. In their respective disengaged configurations, ends 371,373 are released from their respective engagement with locks 384,386, respectively, such that memory recovery force from assembly 370 within lumen 381 forces end 371 out of slit 382, and forces end 373 out of slit 386.

In continued disengagement of lock assemblies 384,388, AAA support assembly 370 continues to unravel through slits 382,386 and around AAA 10.

FIG. 21 shows AAA support assembly 370 and delivery sheath 380 positioned along an anterior aspect of AAA 10. According to the present embodiment and this mode of delivery and use, it is to be appreciated that delivery sheath 380 may not generally require substantial repositioning for complete deployment of AAA support member 370 around AAA 10 according to prior embodiment(s). This is due to the bilateral deployment via slits 382,386 for opposite ends 371,373 of AAA support member 370 to deploy circumferentially around AAA 10. Following full or even partial deployment in this manner, sheath 380 may be longitudinally withdrawn for complete release and implantation of AAA external support assembly 370 around AAA 10.

Thus, the present bi-laterally deployable embodiment is highly beneficial for an anterior approach for delivery and deployment around a AAA, as shown in FIG. 21.

Either or both of the adjustable lock assemblies 384,386 may be adjusted between engaged and disengaged configurations during deployment of AAA support assembly 370, in order to control the deployment in a managed fashion. Many different particular mechanisms may be employed to achieve such adjustable lock, and may be integrated mechanism within the walls of delivery sheath 380 as shown in illustrative schematic shadow, such as deployable and retractable needles for example similar in some regards to the embodiments of FIGS. 5-7B.

Or, other mechanisms may be used as would be apparent to one of ordinary skill based upon review of this disclosure and other available information. For example, a rotatable mechanism, e.g. similarly slotted coaxial member (not shown) may be provided for further example within or around sheath 380. Such may be selectively rotated relative to sheath 380 and slits 382,386 in order to achieve either a disengaged keyed relationship between the adjacent slits, or an engaged relationship via a skewed positioning of the respective slits to provide a normal force of engagement on the AAA support assembly 370.

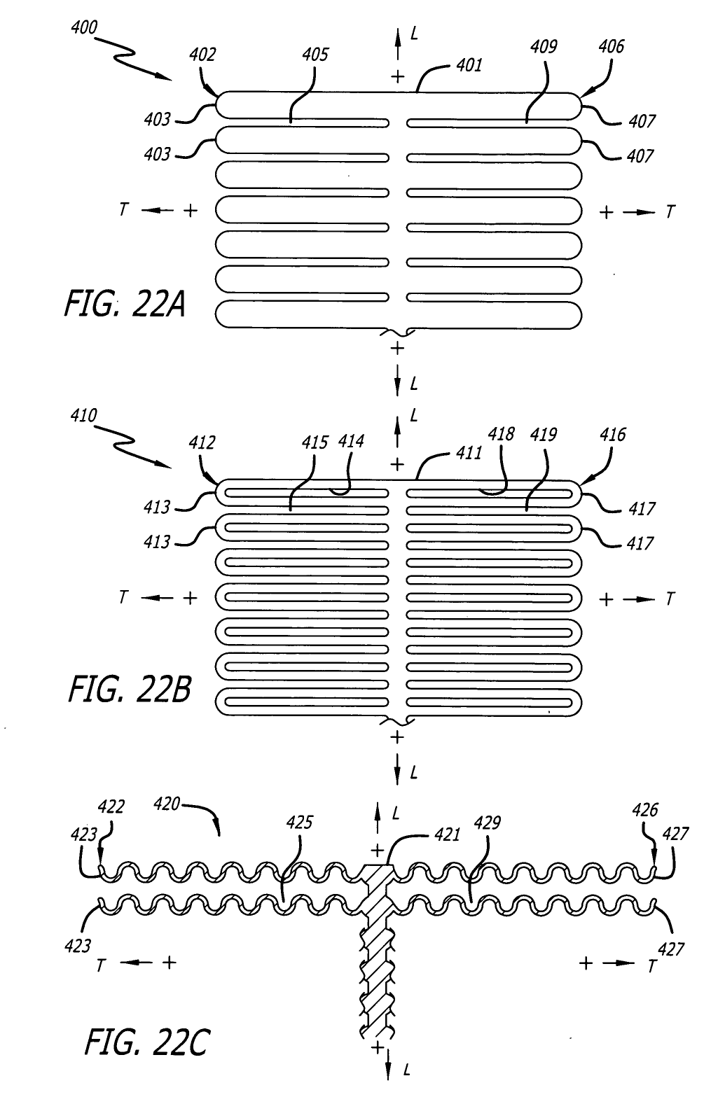

Various support structures may be suitably used according to the anterior delivery embodiments shown and described by reference variously to FIGS. 19A-21. Certain illustrative embodiments are herein shown in FIGS. 22A-C as follows, which are shown in top plan view with an illustrative planar configuration of the respective scaffolds (whereas in use their memory configuration is generally curvilinear about a radius of curvature transverse to the plane of the page). These top views generally show primary planar shapes, whereas the structures may have additionally secondary shapes, such as curvatures down into or out from the page, and furthermore may have tertiary shapes which are shapes within such planes.

FIG. 22A shows a support structure that comprises a solid grated member 400 with a longitudinal spine 401 extending substantially along a longitudinal axis L and substantially intermediate two opposite transverse ends 402,406. Oppositely oriented arrays of lateral support struts or fingers 403,407, respectively, extend laterally away from spine 401 and terminate at opposite transverse ends 402,406, respectively. The respective fingers within the opposite arrays of fingers 403,407 are separated by spaces 405,409, respectively. This configuration is similar to that shown in FIGS. 8-10, except that the spine 401 is located intermediate the two opposite transverse ends 402,406 to accommodate the anterior delivery with bilateral deployment around AAA 10.

FIG. 22B shows a support structure that comprises a grated member 410 with a longitudinal spine 411 extending substantially along longitudinal axis L and substantially intermediate two opposite transverse ends 412,416.

Oppositely oriented arrays of lateral support struts or fingers 413,417, respectively, extend laterally away from spine 411 and terminate at opposite transverse ends 412,416, respectively. The respective fingers within the opposite arrays of fingers 413,417 are separated by spaces 415,419, respectively. This configuration is similar to that shown in FIG. 22A, except that in the present embodiment fingers 413,417 are not solid but include voids therein, such as shown at 414,418, respectively. This configuration provides, among other benefits, for more flexibility along fingers 413,417.

FIG. 22C shows a support structure that comprises a grated member 420 with a longitudinal spine 421 extending substantially along longitudinal axis L and substantially intermediate two opposite transverse ends 422,426.

Oppositely oriented arrays of lateral support struts or fingers 423,427, respectively, extend laterally away from spine 421 and terminate at opposite transverse ends 422,426, respectively. The respective fingers within the opposite arrays of fingers 423,427 are separated by spaces 425,429, respectively. This configuration is similar to that shown in FIGS. 22A-B, except that in the present embodiment fingers 423,427 are undulating, serpentine tertiary shaped members to further enhance flexibility (e.g. similar to certain peak-and-valley patterned designs of certain implantable stents).

This configuration provides, among other benefits, for more flexibility along fingers 423,427.

One or more of the foregoing specific scaffold structures shown in FIGS. 22A-C may be combined with other structures to meet a particular need, such as for example coupling the grated members shown and described with graft materials. Or, they can be combined in various regards along an overall assembly, such as for example at different locations along the assembly. In one particular regard, more flexibility may be required at the longitudinal ends of the scaffold, and at the transverse ends, to accommodate tissue-device transition considerations, e.g. to minimize tissue erosion at transition zones between supported and non-supported tissue, etc.

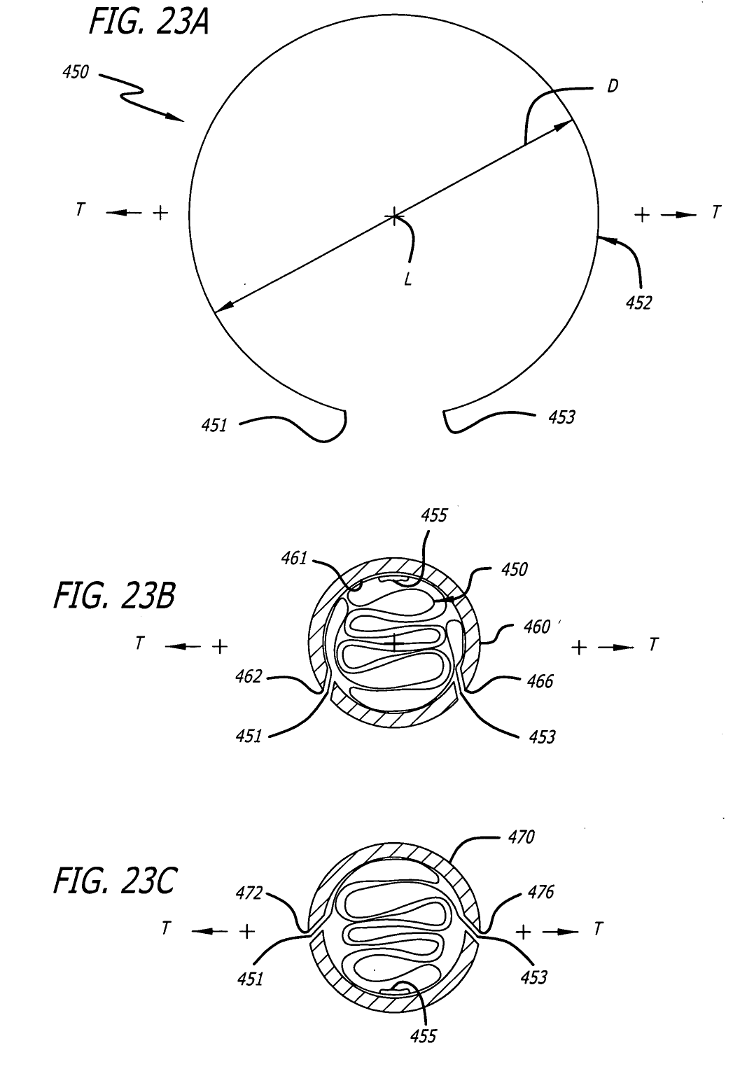

In addition, specific design considerations of the interactive multi-component system may be modified to suit the particular needs of a particular embodiment in use. One particular example is illustrated by reference to FIGS. 23A-C as follows.

FIGS. 23A-C illustrate a further embodiment of the invention as follows.

As shown generally in FIG. 23A in a deployed configuration, an external AAA support assembly 450 includes an adjustable wall 452 with first and second transversely opposed ends 451,453, respectively. As shown in FIG. 23A, AAA support assembly 450 has a memory condition that at least partially circumscribes a diameter D transverse to a longitudinal axis L, such that ends 451,453 face each other relative to the circumference around axis L.

AAA support assembly 450 is adjustable to a first configuration shown in FIG. 23B and that constitutes a deformed configuration from the memory condition shown in FIG. 23A as follows. Assembly 450 is forced into a radially confining lumen 461 of delivery sheath or member 460, and thus is deformed into a deformed shape, which in the particular illustrative embodiment shown in FIG. 23B is in a folded, undulating configuration. Sheath 460 also includes first and second slits 462,466 through which transverse ends 451,453 are engaged, respectively. More specifically, these slits 462,466 are angled in such a manner providing a posterior launch of ends 451,453 down and bilaterally over the right and left sides of AAA 10 during an anterior delivery position of the overall system. This differs for example from prior embodiment of FIG. 21, but shares some similarity with that shown in FIG. 20B—these may be respectively combined or replaced by each other between the embodiments and are not exclusive to one particular combination of other components as provided by the illustrative figures. As previously disclosed for other bilateral deployment arrangements, first and second adjustable lock assemblies (not shown) may also be employed, such as each being adjustable between an engaged configuration and a disengaged configuration relative to the respective AAA support assembly, as previously described above for controllable deployment around AAA 10.

Each of the embodiments shown in FIGS. 23B and 23C include similar components, but differ in the respective arrangements between those components in a manner impacting delivery as follows (and in particular relation to the location of relatively stiffer spine member 455 in the folded, deformed configuration of the overall assembly relative to the angulated, directional delivery slits during delivery and prior to deployment).

FIG. 23B shows AAA support assembly 450 folded in a configuration with folds stacked transverse to transverse axis T, with the exception of opposite end portions that loop to terminate at ends 451,453 in a downward, posteriorly oriented manner seated within posteriorly angulated slots 462,466, respectively. According to a particular embodiment similar to that shown in FIGS. 22A-C, a spine member 455 is located at an anterior aspect along the folded arrangement, substantially opposite the posterior direction. The folded configuration is arranged such that the deformed radius of curvature along spine 455 within lumen 461 is maintained in a similar direction to the radius of curvature at or adjacent to spine 455 under the memory condition, e.g. shown in FIG. 23A. Thus, memory recovery forces may be well harnessed in this configuration with respect to self-applied force to deploy through slits 462,466 when released. Moreover, even superelastic alloys have failure points when subjected to too much strain. Deforming the spined structure in this manner for delivery prior to deployment around a AAA is believed to minimize the strain put onto the material, in particular around the relatively stiffer spine region, when compared to a folded configuration that deforms the assembly against the radius of curvature (FIG. 23C).

More specifically, FIG. 23C shows a similar arrangement to that just described for FIG. 23B, except that the folded deformed configuration for AAA support member 450 differs with spine 455 located on the posterior aspect of the delivery sheath 470 when positioned for deployment. As with other embodiments, sheath 470 includes two opposite transverse slits 472,476 for bilateral stent-graft deployment. However, here spine 455 and adjacent scaffold structure is deformed against its radius of curvature. Though subject to more strain than in the immediate preceding embodiment, the force of recovery to the memory condition may be stronger due to this mode deformation. To the extent strain is not applied to the point of material failure or fatigue in this configuration, this may be a highly beneficial mode in some instances.

The preceding illustrative embodiments variously describe examples of controllable delivery and deployment mechanisms transversely through variously slotted, adjustable delivery sheaths. It is apparent that, in particular in combination with selectively adjustable control mechanisms such as adjustable engaging lock assemblies or other control mechanisms, the ability to control delivery, and at times retract and re-deploy or abort the case, may be highly advantageous. Moreover, one or more of the folded delivery and deployment configurations herein shown and described may also be used in combination with a delivery sheath that is not slotted for transverse delivery purposes. Once properly positioned along a AAA, longitudinal removal of such an outer confining sheath may be sufficient to allow suitable release of the support scaffold for appropriate reconfiguration to the memory condition and associated seating around the AAA in a proper, anatomically conformed manner.

Still further, it is also appreciated that the free opposite transverse ends of the various AAA support scaffold embodiments herein shown and described, and resulting partial unclosed circumferential scaffold, allows for adjustable diameter scaffolding based upon relative distance of the transverse ends from each other when seated around a AAA. While one size may work for most people with most anatomies, it is also contemplated that a range of sizes may be provided for differing anatomies, and may be varied as to recovery diameter, and/or length, and/or stiffness or arrangement of the support scaffold splines, etc.

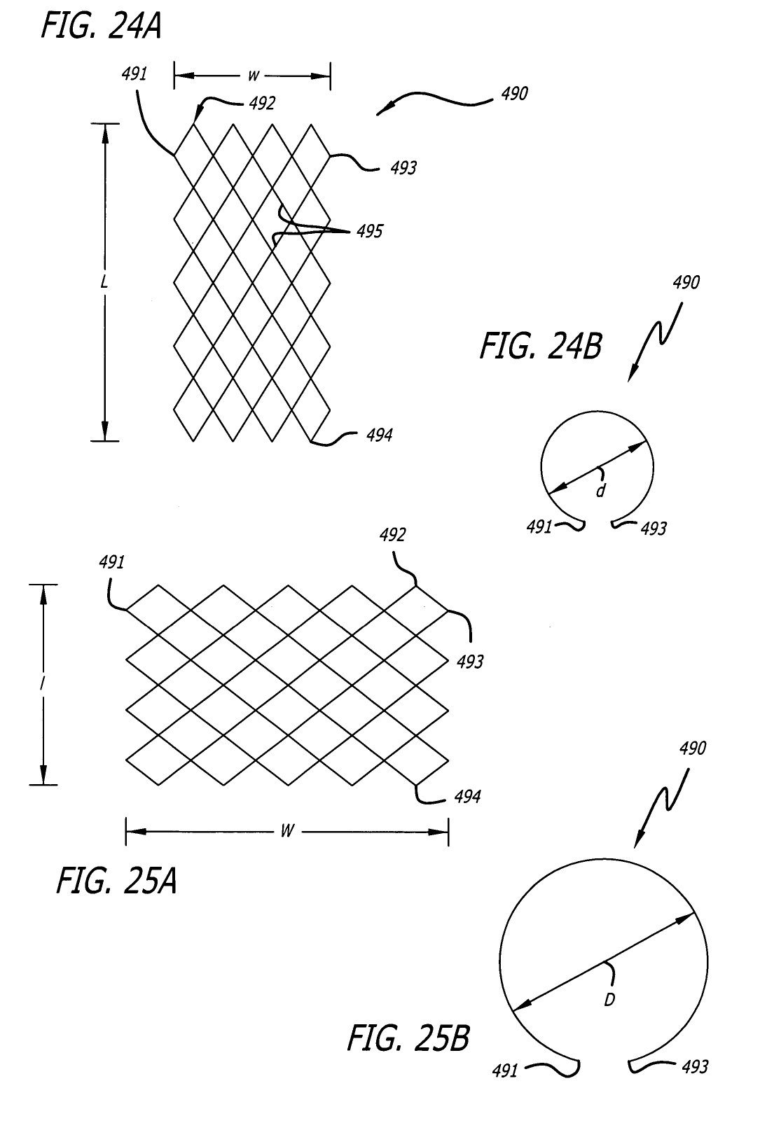

Various modifications of the foregoing may be made without departing from certain broad aspects of the invention. For example, further specific support scaffolds may be used, either instead of or in combination with others herein described. One particular example is illustrated in FIGS. 24A-25B as follows.

FIG. 24A shows a plan view of a more traditional stent scaffold design of networked struts, and may be closed cell of open cell configuration, and may be cut from a tube, or from a sheet, or constructed from formed rings or other discrete strut or patterned portions welded together, etc., as would be apparent and well known to one of ordinary skill. In the present embodiment, unlike most stents used to provide endolumenal scaffolding, assembly 490 is not a solid tubular wall member, but is instead shown flat and discontinuous around a circumference, with two opposite transverse ends 491,493, and two opposite longitudinal ends 492,494. Again, as noted for embodiments shown in FIGS. 22A-C, the planar configuration shown is for illustration, and during intended use a memory condition is typically curvilinear about a radius of curvature that is transverse to the plane of the illustrative page and around the longitudinal axis. As shown in FIG. 24B, the configuration of 24A provides for a first transverse width w and longitudinal length L for deployment, which translates to an available curvilinear or circumferential shape that circumscribes a relatively small diameter d, and thus an ability to provide for a relatively compact rolled or folded configuration for in-vivo delivery in preparation to deployment around a AAA.

As shown in FIG. 25A, the assembly 490 is adjusted to a second configuration with an expanded transverse width W that is larger than width w associated with the first configuration for compact delivery in a collapsed, folded or rolled shape. A shortened longitudinal length I may also result, as also shown in FIG. 25A, and the overall reconfiguration is a result of changing angulation between struts of the network. As a result, assembly 490 is adjusted by memory recovery to a larger circumference, and thus able to self-deploy around a AAA when deployed according to the various deployment schemes herein described or suggested.

The stent-like scaffolding just described may be suitable alone for supporting an early diagnosed AAA, or may be combined with other features such as for example a coupled graft to form a stent-graft composite.

However, such composite combinations may deteriorate the ability to elastically reconfigure in the dramatic fashion that may otherwise be available for example by a nickel-titanium or other superelastic or shape-memory alloy.

In this regard, either elastic, superelastic, or shape-memory modes of materials or alloy reconfiguration may be employed according to the various embodiments herein shown and described.

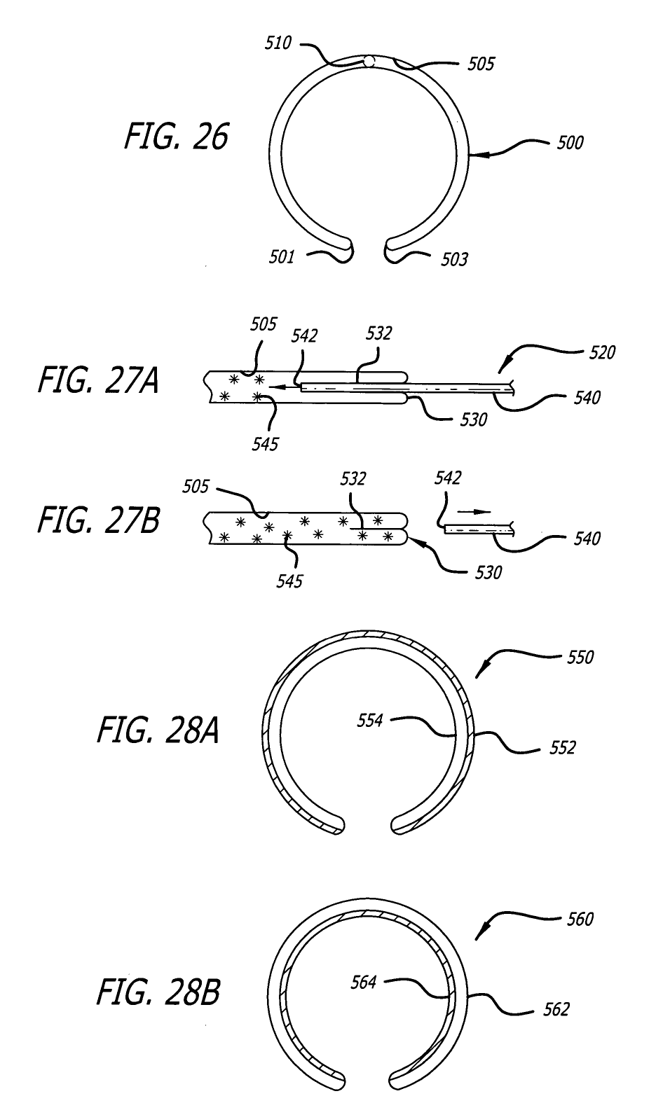

For the purpose of still further illustrating the broad intended scope of various aspects hereunder, the embodiments of FIGS. 26-28B show and describe various inflatable balloon or bladder modes of achieving one or more intended results hereunder.

More specifically, FIG. 26 shows a cross-sectioned schematic representation of an inflatable bladder 500 as a support scaffold. Bladder 500 includes a curved wall with two transversely opposed ends 501,503 similar to other embodiments, except that bladder 500 includes a bladder chamber 505 that is adapted to be coupled to a source of pressurizeable fluid via a coupling 510, shown schematically in shadow in FIG. 26 and further developed below.

In one further particular embodiment shown in FIG. 27A, coupling 520 includes a self-sealing valve assembly 530, shown as for example a “duck-bill” type of valve, which includes a self-sealing pore or bore 532 through which a removable needle 540 is removably engaged. When needle 540 is engaged within valve bore 532 of valve assembly 530 as shown in FIG. 27A, fluid agent 545 may be injected from needle tip 542 to fill and pressurize bladder chamber 505. This process brings bladder 500 from an unpressurized and “flaccid” configuration suitable for folding and minimally invasive delivery through a sheath, to a pressurized and taut configuration heavily influenced by a memory shape of the bladder wall material (as shown in the desired shape for external AAA support scaffold in FIG. 26). By removing needle tip 542 from bladder chamber 505 and withdrawal of needle 540 from valve 530 following pressurization of bladder chamber 505, valve 530 self seals under the internal pressure of bladder chamber 505. While this particular self-sealing mechanism is considered appropriate for many specific applications, it is to be appreciated as an illustrative example, and other forms of bladder inflation and sealing may be used in the alternative to, or addition to, this specific embodiment.

Various material processing techniques, and resulting structures, may be employed to achieve the shaped deployable bladder scaffold just described, or modify it to further improved modes. For example, as shown in FIGS. 28A-B, further inflatable bladder embodiments 550,560 may include an outer radial surface 552,562, respectively, that is of different material property than the inner radial surface 554,564, also respectively. By providing for example such surface with more or less compliance than the opposite surface, re-configuration to differing shapes under applied internal bladder pressure is effected. Moreover, such surfaces may be treated to meet particular tissue-interface needs. For example, the inner radius portions 554,564, respectively, directly interface with (or at least face) the outer surface of the AAA to be supported. Therapeutic agents as herein described may be provided on that surface, and may include for example a surface similar to or the same as those described by reference to FIGS. 16-18. Or, the external radius surfaces of these embodiments 552,562 may also benefit from active agent, e.g. to prevent biologic rejection, sepsis, uncontrolled scarring, etc., or to promote cellular deposition and biologic acceptance.

In this regard, the various tissue interface surfaces of mechanical scaffolds herein described may use various forms of polymer agents, and in particular biopolymer agents, that may improve biologic acceptance of the implant, and possibly improve and/or possibly even regenerate the target aneurismal wall tissue. Such may be adhered to the surface for simple biologic surface interaction, or may elute therefrom. In this regard, as elsewhere herein noted, certain injectable polymer agents, such as fibrin glue agent, may be used on its own as a direct scaffold with direct therapeutic effect on vascular aneurysms in particular. Further more detailed examples of such injectable polymers are described as follows.

In general, a “polymer” is herein defined as a chain of multiple units or “mers”. Fibrin glue for example contains polymerized fibrin monomers, and is further herein considered an illustrative example of a biopolymer since its components are biological.

Fibrin glue is an already FDA approved biomaterial that is routinely used as a surgical adhesive and sealant. This biopolymer is formed by the addition of thrombin to fibrinogen. Thrombin in a kit is an initiator or catalyst which enzymatically cleaves fibrinogen which alters the charge and conformation of the molecule, forming a fibrin monomer. The fibrin monomers then proceed to aggregate forming the biopolymer fibrin. After combination of the two thrombin and fibrinogen components, the solution remains liquid for several seconds before polymerizing. According to certain further embodiments of the present invention, fibrin glue agent, either immediately following mixture of the precursor materials, or by delivering the materials separately to mix in-situ, is therefore adapted to be delivered to the aneurysm wall to provide a support scaffold there, such as via injection catheters or other injectors, thus requiring only a minimally invasive procedure. It is also biocompatible and non-toxic, without inducing inflammation, foreign body reactions, tissue necrosis or extensive fibrosis.

Native fibrin is highly involved in wound healing and acts as the body's natural matrix for angiogenesis. Endothelial cells migrate through the fibrin clot via alphaᵥbeta₃ integrin binding to RGD motifs in fibrin. Production of plasmin at the location of migrating endothelial cells degrades the fibrin matrix. This decrease in fibrin density allows for capillary tube formation. As the cells migrate through the less dense fibrin, they interact with residues on the beta-chain of fibrin via vascular endothelial cadherins and promote capillary morphogenesis. In addition to providing a matrix for endothelial cell migration and capillary tube formation, fibrin also acts as a sustained release reservoir for several growth factors and fibrinolytic enzymes. A degradation product of fibrin, fibrin fragment E, is also characterized and observed to:

induce angiogenesis; stimulate proliferation, migration and differentiation of human microvascular endothelial cells; and stimulate migration and proliferation of smooth muscle cells. Fibrin glue is also believed to up-regulate or release various growth factors, which may recruit other cells to the aneurysm or inhibit the processes of aneurysm progression. Fibrin glue has been observed to induce fibroblast migration and may cause recruitment and proliferation of fibroblasts in a cardiac infarct, resulting in a thicker infarct wall.

It is also possible that injection of fibrin glue results in recruitment of stem cells from the bone marrow, which may aid in new vessel development.

Further more detailed examples of fibrin glues that may be useful according to various aspects of the present invention are disclosed in the following reference: Sierra, DH, “Fibrin sealant adhesive systems: a review of their chemistry, material properties and clinical applications.” J Biomater Appl. 1993;7:309-52. The disclosure of this reference is herein incorporated in its entirety by reference thereto.

According to still a further embodiment of the invention, a preparation of living material, such as for example cells, in combination with. a non-living material is delivered into, or exteriorly on, an aneurysm (e.g. in particular a thoracic or abdominal aneurysm) to form a scaffolding there. In one further more detailed embodiment, the polymeric material is adapted to enhance retention of the cells being delivered into the location where the scaffolding is to be formed. In another regard, the polymeric material is adapted to further contribute to forming the scaffolding, such as by providing wall support via the polymerized chain of material within the region.

One particular example of a material that provides significant benefit in such combination with cellular therapy is fibrin glue, as elsewhere herein noted. More specifically, fibrin glue has been observed to provide enhanced retention of cells such as myoblasts or fibroblasts that are injected into cardiac tissue in order to treat damaged cardiac structures, such as infarct regions of a heart.

Notwithstanding the significant benefit of using fibrin glue in combination with cell delivery for treating vascular aneurysms, other suitable materials having beneficial effects in such combination are also contemplated, such as other polymers or molecular scaffolds or materials that impact the extracellular matrix in vascular wall tissue structures to substantially enhance function and/or support of a damaged or degraded wall. Moreover, collagen or precursors or analogs or derivatives thereof may be further considered useful for this purpose, either in addition or in the alternative to fibrin glue.

Embodiments of injectable scaffolding material according to the invention may include primarily or only one injectable scaffolding material, or may include combinations of materials. For example, embodiments of injectable scaffolding material that includes cells may include other materials, such as fluids or other substrates to provide the cells in an overall preparation as a cellular media that is adapted to be injected, such as in particular through a delivery lumen of a delivery catheter. In one particular example that has been observed as useful, the injectable scaffolding material may include skeletal myoblasts or other suitable substitute cells in combination with a biopolymer agent such as fibrin glue agent, which may itself be provided as two precursor materials that are mixed to form fibrin glue that assists in forming a scaffold when delivered with cells at the desired location associated with a vascular aneurysm.

Notwithstanding the substantial benefit that may be gained from such specialized tools and techniques to meet particular needs as described herein, such particular modes for forming injected vascular aneurysm wall scaffolds, or otherwise conducting cell therapy for treating or preventing aneurysms, are not to be considered limiting to the various broad aspects of the present invention.

For example, it is to be appreciated that fibrin glue expresses several different modes of beneficial bioactivity that each provides or enhances particular therapeutic results of the fibrin as an applied vascular wall scaffold for therapy of aneurysms. Accordingly, the fibrin agent itself is an illustrative mode of such bioactive features as broader aspects having independent value (despite the additional value from the various combinations of features). In one regard, fibrin includes RDG binding sites which have been observed to increase affinity of cells into the area, including cell delivered with the fibrin or recruited into the area. In addition, fibrin includes a fragment “E” which has been observed to induce angiogenesis. Each of these represents an independent benefit of fibrin glue as a scaffold for cell therapy, and their combination is in particular further beneficial. For example, the cell affinity provided by the RDG binding sites allow a cellular matrix to form within the scaffolding at an injected region, whereas the angiogenesis from the fragment E allows for longevity and viability of the cellular matrix via induced blood supply. This is in particular beneficial for example in applications injecting the scaffolding into or onto damaged vascular wall tissue, and in particular to treat aneurysms, enhancing the vascular wall while preventing negative remodeling that may otherwise progress without the long-term cell viability in the area.

Accordingly, the fibrin glue is to be considered illustrative of the features which provide these benefits, and other modifications may be made in further embodiments providing other injectable compounds for similar activities. For example, injecting a material into or onto vascular wall tissues as described and that express RDG binding sites in a resulting injected scaffold is a broad aspect of the invention illustrated but not limited to the particular beneficial embodiment of fibrin glue. In another example, injecting a polymer agent into or onto weakened or aneurismal vascular wall tissue in a manner which induces angiogenesis is another broad aspect illustrated by the fibrin glue but not necessary limited to that particular beneficial embodiment in all cases. In particular, modifications of the detailed embodiments may include other molecular forms which provide fragment E than specifically via fibrin molecules. Still further, the combination of RDG binding activity (or other cellular affinity factors) and fragment E (or other angiogenic factors) may be achieved in other manners than specifically via fibrin without departing from such various broad aspects of the invention.

Notwithstanding the foregoing statements intended to remove the limitation of fibrin glue from certain broad aspects of the invention, it is nevertheless to be appreciated that fibrin glue does provide tremendous value and benefit in its own regard, such as by individually providing the combination of features and benefits just described as an injectable scaffold agent.

Other polymers or molecular scaffolds or materials, which may be injectable themselves or in the form of precursor agents, are briefly described as follows. Several synthetic polymers, such as polyethylene oxide (“PEO”), PEO-poly-l-lactic acid (“PLLA-PEO block copolymer”), poly(N-isopropylacrylamide-co-acrylic acid) (“poly(NIPAAm-co-Aac)”), pluronics, and poly-(N-vinyl-2-pyrrolidone) (“PVP”) may be adapted to provide artificial extracellular matrices for transplanted cells, or otherwise provide support scaffold into or on weakened or aneurismal vascular wall tissue. Various biologic polymers such as alginate, collagen, and of course fibrin glue, may be prepared in a manner for use as injectable scaffolds in certain settings.

Benefits of each of these polymers include that they may be injected into the desired location without the need for more invasive implantation.

In one more specific example, PEO is generally considered biocompatible and is known not to react with proteins and most biologic macromolecules. It is injectable, though larger needles such as 22 gauges are generally to be used for this material. According to another example, PEO-PLLA-PEO block copolymers are also generally considered biocompatible and biodegradable. However, formulations with this compound will typically undergo gel solution transitions around about 45° C., and thus are typically to be injected at temperatures above body temperature. A respective treatment system would in such circumstance generally also include a heater assembly. Poly(NIPAAm-co-AAc) gels also undergo gel solution transitions, which gels generally remain liquid at room temperature and solidify at body temperature. In order to have a mechanically stable gel, larger gauge needles may also be particularly useful. Pluronics are also known to be generally biocompatible, but are not typically considered biodegradable. They remain liquid at temperatures lower than 4° C., and thus catheter or other form of delivery tool may also further include active cooling and/or insulation along the catheter to provide and maintain the material at such temperatures until delivered. PVP is a material that may be injected through smaller gauge needles or devices, e.g. as small as 30 gauge. It is also generally non-antigenic and non-toxic; however, it is generally not considered biodegradable. Alginate gels are typically linked together by calcium ions, which will dissociate and render the gel mechanically unstable over a period of time. They are also generally considered non-biodegradable and have been observed to be immunogenic in certain settings. Collagen gels are generally considered biocompatible and biodegradable, but are not typically mechanically stable.

Certain additional materials have been disclosed for use to form sponges as scaffolds for cell culture and transplantation. In one particular series of disclosures, polysaccharide sponges are intended to be applied in such a manner. However, these disclosures have not suggested suitable modifications of these structures to provide for an injectable or otherwise deliverable scaffolding agent well suited for delivery via needle injection or transcatheter techniques, nor have they been generally used for treating aneurismal or otherwise weakened vessel walls, such as for example but without limitation thoracic or abdominal aortic aneurysms. Nevertheless, where possible it is herein contemplated to make such modifications for delivery of molecular scaffolds as further aspects hereunder.

Further more detailed examples of various aspects of the materials described immediately above are provided in one or more of the following references: MERRILL EW. “Poly(ethylene oxide) star molecules: synthesis, characterization, and applications in medicine and biology,” J Biomater Sci Polym Ed, 1993;5:1-11; PEPPAS NA, Langer R. “New challenges in biomaterials,” Science, 1994;263:1715-20; SIMS C D, Butler P E, Casanova R, Lee B T, Randolph M A, Lee W P, Vacanti C A, Yaremchuk M J, “Injectable cartilage using polyethylene oxide polymer substrates,” Plast Reconstr Surg. 1996;98:843-50; JEONG B, Bae Y H, Lee D S, Kim S W, “Biodegradable block copolymers as injectable drug-delivery systems,” Nature, 1997;388:860-2; STILE RA, Burghardt W R, Healy K E, “Synthesis and Characterization of Injectable Poly(N-isopropylacrylamide)-Based Hydrogels That Support Tissue Formation in Vitro,” Macromolecules, 1999;32:7370-7379; ARPEY C J, Chang L K, Whitaker D C, “Injectability and tissue compatibility of poly-(N-vinyl-2-pyrrolidone) in the skin of rats: a pilot study,” Dermatol Surg, 2000;26:441-5; discussion 445-6; SMIDSROD O, Skjak-Braek G. “Alginate as immobilization matrix for cells,” Trends Biotechnol, 1990;8:71-8; Paige K T, Cima L G, Yaremchuk M J, Vacanti J P, Vacanti C A. “Injectable cartilage,” Plast Reconstr Surg, 1995;96:1390-8; discussion 1399-400. The disclosures of these references are herein incorporated in their entirety by reference thereto.

Various of the materials described herein are considered useful according to various of the present embodiments, either alone or in combination or blends with others, such as for example in addition or in the alternative to fibrin glue. These compounds also illustrate certain broader classes of compounds, which classes may contribute additional alternatives as would be apparent to one of ordinary skill. Moreover, the compounds listed may be delivered to tissue by delivering precursor materials to the tissue which form the intended compound in situ. For example, alginate is an illustrative form of polymerized polysaccharide which may be suitably prepared for injection and provide various of the benefits herein described. In one particular example, alginate as a polymer may be made injectable for example by varying the concentration of the polysaccharide and calcium. Such preparation, or other injectable preparation, may be thus injected into or onto weakened or damaged vessel walls according to various aspects described herein, again either instead of or in combination with fibrin glue or other compounds as would be apparent to one of ordinary skill.

Moreover, whereas polymers are in particular beneficial means to provide scaffolding to vessel walls and supporting cell therapy, other types of materials than polymers may be used according to various aspects of the invention and thus represent further contemplated embodiments. For example, integrin is an example of a protein which has been observed to enhance cellular binding and thus may be injected into aneurysms to provide substantial benefit to cellular tissue formation and/or retention there. For further illustration, further particular embodiments may also include integrin in combination with cell delivery, and/or in combination with others of the non-living compounds herein described as useful according one or more of the aspects of the invention.

In comparison with the foregoing list of exemplary polymers and other potential injectable scaffolding agents, it is nevertheless appreciated that fibrin glue provides a valuable and relatively unique combination of benefits in that it is generally considered biocompatible, non-toxic, and biodegradable; it may also be injected through 30 gauge needles at room or body temperature.

Moreover, it provides the combination of bioactivities providing combined therapy as injectable scaffold which many other agents are not suited to provide.

It is still to be appreciated, however, that where fibrin glue or related agents are herein described, it is further contemplated that other materials such as collagen, or precursors or analogs or derivatives thereof, may also be used in such circumstances, in particular relation to forming injected scaffolding, either alone or in combination with cells.

Moreover, where a compound is herein identified in relation to one or more embodiments described herein, such as for example collagen or fibrin, precursors or analogs or derivatives thereof are further contemplated. For example, material structures that are metabolized or otherwise altered within the body to form such compound are contemplated. Or, combination materials that react to form such compound are also contemplated. Additional materials that are also contemplated are those which have molecular structures that vary insubstantially to that of such designated compounds, or otherwise have bioactivity substantially similar thereto with respect to the intended uses contemplated herein (e.g. removing or altering non-functional groups with respect to such bioactive function). Such group of compounds, and such precursors or analogs or derivatives thereof, are herein collectively, or individually, referred to as “compound agent(s).”

Similarly, reference herein to other forms of “agents”, such as for example “polymer agent” or “fibrin glue agent” may further include the actual final product, e.g. polymer or fibrin glue, respectively, or one or more respective precursor materials delivered together or in a coordinated manner to form the resulting material.

In a still similar regard, the term “agent” is herein intended to have similar meaning to mechanical structures, wherein an “agent” may have a certain functional property in one mode, condition, or configuration, but may not always be provided in that context and may be suitably modified or adjusted to that context. For example, a “scaffold agent,” “support agent,” or words of similar import or combinations thereof, may include one or more mechanical, structural, or molecular components that are adjustable or combinable to provide the intended final result in terms of the related ultimate structure or related function. For purpose of further illustration, a “support scaffold agent” may include for example a stent-graft assembly that is adapted to provide an external aneurysm support scaffold in one configuration, condition, or shape, but may be deliverable in another configuration, condition, or shape not suitable in itself to provide such functionality. In either case, such stent-graft assembly would be considered a support scaffold agent within the intended meaning. In another illustrative example, a polymer agent provided in two separate precursor parts may also be a “support scaffold agent” though support scaffolding may only be achieved upon their mixing and polymerization in-situ on a tissue wall.

Various further systems, materials, devices, and methods have been disclosed for use as cardiac chamber scaffolding to provide therapy for weakened or otherwise dysfunctional heart tissue. It is to be appreciated that such approaches for heart therapy may be suitably modified upon review of the totality of this disclosure for use in providing external vascular aneurysm scaffolding support.

Previously disclosed tissue engineering approaches for cardiac therapy are generally intended to repair lost or damaged tissue through the use of cellular transplantation, mechanical, and biomaterial scaffolds. Several groups have disclosed methods intended to improve cardiac function through the injection of cells alone into ischemic myocardium. One group also disclosed suturing fetal cardiomyocyte-seeded alginate gels to the epicardial surface in order to preserve LV function.

Several prior attempts have been disclosed with the intended purpose of providing mechanical external constraints as external support to limit negative left ventricular remodeling, which is believed to contribute independently to the progression of heart failure following a myocardial infarction.

One previously disclosed study included suturing a polymeric mesh to the epicardial surface for the intended purpose of providing an external support to prevent LV dilation and deterioration of LV function post-MI.

Another previously disclosed device that has been investigated provides a plurality of sutures that are implanted in an open-chest procedure across the ventricle under tension to provide a change in the ventricle shape and a decrease chamber diameter. This trans-cavitary suture network is intended to decrease the radius of the ventricle to thus reduce ventricular wall stress.

Another previously disclosed device under clinical investigation is generally a mesh structure that is implanted as a jacket around the heart and adjusted to provide a snug fit during open-chest surgery. It is intended that the jacket restrains the heart from further enlargement. Still another approach being investigated provides a nitinol mesh as, in some regards, a similar external restraining device to that described above. However, the super-elastic system is intended to assist in systolic contraction, and is generally intended for use via thorascopically guided minimally invasive delivery. Still another system being investigated includes a rigid ring that is implanted during open-chest surgery as another external constraining device to the ventricle. This ring is intended to decrease ventricular wall stress and prevent further enlargement of the heart by reducing the radius and modifying the shape of the ventricle. Yet another device approach that was at one time being investigated includes a radiofrequency (“RF”) ablation catheter intended to shrink damaged, i.e. infarcted scar, tissue during cardiac surgery.

Additional examples of devices and methods similar to one or more of those discussed above have been disclosed by various companies, including the following: “Acorn;” “Myocor;” “Paracor;” “Cardioclasp;” and “Hearten.”

The following issued U.S. Patents are herein incorporated in their entirety by reference thereto: U.S. Pat. No. 6,077,218 to Alferness; U.S. Pat. No. 6,085,754 to Alferness et al.; U.S. Pat. No. 6,123,662 to Alferness et al.; U.S. Pat. No. 6,126,590 to Alferness; U.S. Pat. No. 6,155,972 to Nauertz et al.; U.S. Pat. No. 6,165,121 to Alferness; U.S. Pat. No. 6,165,122 to Alferness; U.S. Pat. No. 6,169,922 to Alferness et al; U.S. Pat. No. 6,174,279 to Girard; U.S. Pat. No. 6,179,791 to Krueger; U.S. Pat. No. 6,193,648 to Krueger; U.S. Pat. No. 6,230,714 Alferness; U.S. Pat. No. 6,241,654 to Alferness; U.S. Pat. No. 6,293,906 to Vanden Hoek; U.S. Pat. No. 6,370,429 to Alferness; U.S. Pat. No. 6,375,608 to Alferness; U.S. Pat. No. 6,416,459 to Haindl; U.S. Pat. No. 6,425,856 to Shapland; U.S. Pat. No. 6,482,146 to Alferness et al.; U.S. Pat. No. 6,537,203 to Alferness et al.; U.S. Pat. No. 6,564,094 to Alferness et al.; U.S. Pat. No. 6,567,699 to Alferness et al.; U.S. Pat. No. 6,572,533 to Shapland et al.; U.S. Pat. No. 6,575,921 to Vanden Hoek et al.; U.S. Pat. No. 6,579,226 to Vanden Hoek et al.; U.S. Pat. No. 6,582,355 to Alferness et al.; U.S. Pat. No. 6,587,734 to Okuzumi.

The following Published U.S. Patent Applications are also herein incorporated in their entirety by reference thereto: U.S. Patent Application Nos. 2002/0068850A1 to Vanden Hoek et al.; 2002/0082647A1 to Alferness; 2002/0091296A1 to Alferness; 2002/0103511A1 to Alferness et al.; 2002/0111567A1 to Vanden Hoek et al.; 2002/0133055A1 to Haindl; 2002/0151766A1 to Shapland et al.; 2002/0151950A1 to Okuzumi; 2003/0028077A1 to Alferness et al.; and 2003/0045776A1 to Alferness et al.

The following PCT International Patent Applications are also herein incorporated in their entirety by reference thereto: PCT International Publication Nos. WO/02/38081A3 to Vanden Hoek et al.; WO/02/43617A2 to Vanden Hoek et al.; and WO/02/43617A3 to Vanden Hoek et al.

In addition, the following issued U.S. Patent Nos. are also herein incorporated in their entirety by reference thereto: U.S. Pat. Nos. 5,961,440 to Schweich, Jr. et al.; U.S. Pat. No. 6,045,497 to Schweich, Jr. et al.; U.S. Pat. No. 6,050,936 to Schweich, Jr. et al.; U.S. Pat. No. 6,059,715 to Schweich, Jr. et al.; U.S. Pat. No. 6,077,214 to Mortier et al.; U.S. Pat. No. 6,162,168 to Schweich, Jr. et al.; U.S. Pat. No. 6,165,119 to Schweich, Jr. et al.; U.S. Pat. No. 6,165,120 to Schweich, Jr. et al.; U.S. Pat. No. 6,183,411 to Mortier et al.; U.S. Pat. No. 6,260,552 to Mortier et al.; U.S. Pat. No. 6,261,222 to Schweich, Jr. et al.; U.S. Pat. No. 6,264,602 to Mortier et al.; U.S. Pat. No. 6,332,863 to Schweich, Jr. et al.; U.S. Pat. No. 6,332,864 to Schweich, Jr. et al.; U.S. Pat. No. 6,332,893 to Mortier et el.; U.S. Pat. No. 6,402,679 to Mortier et al.; U.S. Pat. No. 6,402,680 to Mortier et al.; U.S. Pat. No. 6,406,420 to McCarthy et al.; U.S. Pat. No. 6,514,194 to Schweich, Jr. et al.; U.S. Pat. No. 6,537,198 to Vidlund et al.; and U.S. Pat. No. 6,589,160 to Schweich, Jr. et al.

Still further, the following Published U.S. Patent Applications are also herein incorporated in their entirety by reference thereto: U.S. patent application Ser. Nos. 2002/0077524A1 to Schweich, Jr. et al.; 2002/0169358A1 to Mortier et al.; 2002/0169359A1 to McCarthy et al.; 2002/0173694A1 to Mortier et al.; 2003/0032979A1 to Mortier et al., 2003/0050529A1 to Vidlund et al.; and 2003/0130731 to Vidlund et al.

In addition, PCT International Publication No. WO/02/67985A1 to Lau et al. is also herein incorporated in its entirety by reference thereto. Additional PCT International Publication Nos. WO 01/91667A2 to Melvin et al., and WO 01/91667A3 to Melvin et al. are also herein incorporated in their entirety by reference thereto.

Still further, the following U.S. Patent Nos. are also herein incorporated in their entirety by reference thereto: U.S. Pat. No. 5,928,224 to Laufer; U.S. Pat. No. 5,989,284 to Laufer; U.S. Pat. No. 6,071,303 to Laufer; U.S. Pat No. 6,106,520 to Laufer et al.; and U.S. Pat. No. 6,283,935 to Laufer et al.

Also herein incorporated in its entirety by reference thereto is the following published U.S. patent application: WO2004/050013 to Lee et al. as inventors, and The Regents of the University of California as Applicant. In particular, this publication provides certain disclosed systems and methods related to injectable scaffolds to provide support to tissue structures, and in particular polymer agents, and in particular various injection assemblies, that are variously considered useful as applied and suitably modified according to one of ordinary skill to meet the special and unique objectives of the present invention to treat weakened or aneurismal walls of vessels, such as the aorta, or other lumens. In one specific regard, various aspects of the present invention may be suitably accomplished by further incorporation of, or appropriate modifications of, the injection assemblies provided in this incorporated reference. This includes, in particular but without limitation, to the extent adapted to deliver two-part precursor materials in a manner allowing in-situ mixing at or adjacent to the tissue site of delivery within the body. Of still more particular interest to the present invention are such assemblies and methods that are adapted for delivery of two-part polymer agent systems, and/or delivery of living cells in combination with other injectable agents such as polymer agents, for in-situ mixing.

Still further more detailed examples of cardiac tissue conditions, devices and systems intended to provide interventional solutions for various medical conditions, tissue engineering materials and techniques, research tools, and various tissue culturing and intended cellular therapy methods, are variously disclosed in the following references for further background understanding:

1. Taylor D A, et al. Regenerating functional myocardium: improved performance after skeletal myoblast transplantation. Nat Med. 1998;4:929-33.

2. Leor J, et al. “Bioengineered cardiac grafts: A new approach to repair the infarcted myocardium?” Circulation. 2000;102:III56-61.

3. Cleutjens J P, et al., “Regulation of collagen degradation in the rat myocardium after infarction.” J Mol Cell Cardiol. 1995;27:1281-92.

4. Erlebacher J A, et al., “Early dilation of the infarcted segment in acute transmural myocardial infarction: role of infarct expansion in acute left ventricular enlargement.” J Am Coll Cardiol. 1984;4:201-8.

5. Olivefti G, et al., “Side-to-side slippage of myocytes participates in ventricular wall remodeling acutely after myocardial infarction in rats.” Circ Res. 1990;67:23-34.

6. Pfeffer M A, et al., “Ventricular remodeling after myocardial infarction.

Experimental observations and clinical implications.” Circulation. 1990;81:1161-72.

7. Warren S E, et al., “Time course of left ventricular dilation after myocardial infarction: influence of infarct-related artery and success of coronary thrombolysis.” J Am Coll Cardiol. 1988;11:12-9.

8. Hunyadi J, et al., “Keratinocyte grafting: a new means of transplantation for full- thickness wounds.” J Dermatol Surg Oncol. 1988;14:75-8.

9. Horch R E, et al., “Single-cell suspensions of cultured human keratinocytes in fibrin-glue reconstitute the epidermis.” Cell Transplant. 1998;7:309-17.

10. Andree C, et al., “Plasmid gene delivery to human keratinocytes through a fibrin-mediated transfection system.” Tissue Eng. 2001;7:757-66.

11. Sims C D, et al., “Tissue engineered neocartilage using plasma derived polymer substrates and chondrocytes,” Plast Reconstr Surg. 1998;101:1580-5.

12. Bach A D, et al., “Fibrin glue as matrix for cultured autologous urothelial cells in urethral reconstruction.” Tissue Eng. 2001;7:45-53.

13. Han B, et al., “A fibrin-based bioengineered ocular surface with human corneal epithelial stem cells.” Cornea. 2002;21:505-10.

14. Watanabe E, et al., “Cardiomyocyte transplantation in a porcine myocardial infarction model.” Cell Transplant. 1998;7:239-46.

15. Chawla P S, et al., “Angiogenesis for the treatment of vascular diseases.” Int Angiol. 1999;18:185-92.

16. Kipshidze N, et al. “Angiogenesis in a patient with ischemic limb induced by intramuscular injection of vascular endothelial growth factor and fibrin platform.” Tex Heart Inst J. 2000;27:196-200.

17. Sakiyama-Elbert S E, Hubbell J A. “Development of fibrin derivatives for controlled release of heparin-binding growth factors.” J Control Release. 2000;65:389-402.

18. Pandit A S, Feldman D S, Caulfield J, et al. “Stimulation of angiogenesis by FGF-1 delivered through a modified fibrin scaffold.” Growth Factors, 1998;15:113-23.

The disclosures of each of the references provided immediately above, or as elsewhere indicated in this disclosure, are herein incorporated in their entirety by reference thereto.

The disclosures of the following issued U.S. Patents are also herein incorporated in their entirety by reference thereto: U.S. Pat. No. 5,103,821 to King; U.S. Pat. No. 6,151,525 to Soykan et al.; and U.S. Pat. No. 6,238,429 to Markowitz et al.; The disclosures of the following PCT International Patent Application Publications are also herein incorporated in their entirety by reference thereto: WO 90/10471 to King; and WO 98/02150 to Stokes et al.

According to additional aspects and modes of the present invention, various of the previously disclosed systems, materials, and methods for providing scaffolding, angiogenesis and regeneration, and support to other tissue structures, and in particular cardiac tissue structures, are suitably modified in order to provide a substantially new and highly beneficial therapy for vascular aneurysms or otherwise weakened vessel walls. Such may include for example, but without limitation: cell therapy, polymeric therapy, implantation of biological or other manufactured mechanical support scaffolds, or combinations thereof, as would be apparent to one of ordinary skill based upon review of this disclosure and other available information, including without limitation the references herein incorporated in their entirety.

According to the various device implants, e.g. stent-graft embodiments, and injectable agent embodiments herein disclosed to provide certain desirable medical therapeutic results, certain broad aspects of the invention are to be clearly understood. Accordingly, in one regard, it is to be appreciated that each a stent-graft or other form of device scaffold, or injectable agent scaffold, such as polymerizing agents such as fibrin glue, is a distinct form of a broader concept which is a “scaffold agent”, “scaffold member”, or the like. According to certain embodiments therefore, such scaffold agents or members are deliverable to locations adjacent to AAAs in first configurations and are thereafter adjustable to second configurations that provide scaffolding support to the AAA sufficient to substantially prevent further dilation at the scaffolded region.

Further particular benefit is gained by applying the various aspects of the invention as an “outside-in” approach that is removed from the blood pool with substantial benefits stemming therefrom as long-term implants. For example, coumadin or other anti-clofting regimens may not thus be required as is required for endolumenal AAA grafts, with systemic bleeding complications, etc. Still further benefit is afforded by providing such scaffold agents in transthoracic delivery modalities, such as with thoracoscopic assistance or otherwise. Nonetheless, each embodiment or particular mode herein disclosed may provide further particular benefit that should be appreciated as well.

Despite the particular benefits herein described for minimally invasive delivery of external vascular aneurysm scaffolds, it is also appreciated that certain broad aspects of the invention may be achieved according to other modalities. In one regard, various molecular scaffold embodiments herein described are well suited for use together with more traditional endograft devices and techniques, such as in order to maintain a healthier wall behind or adjacent the graft, or to improve graft incorporation to the wall. In another regard, applications of molecular or mechanical agents or scaffolds to the exterior wall surface provides substantial benefits, even if done under direct surgical techniques. In this regard, more traditional direct injectors, sprayers or nebulizers, other coating techniques or devices can be used to directly apply these materials around the outside surface of a weakened or aneurismal vessel or lumen wall. In regards to mechanical scaffolds such as external stent grafts, these may be directly applied around a AAA for example, with the aid of suturing ends together, or otherwise. Such direct access affords various improved forms of the devices and other modes of material delivery that otherwise may be compromised in their functionality by the necessity to accommodate less invasive or minimally invasive delivery tools and techniques through small spaces.

This disclosure variously describes the embodiments in terms of systems, assemblies, or devices for treatment of AAA conditions. While combinations of the components of such embodiments are highly beneficial, it is contemplated that each individual component alone may be highly beneficial, such as for example by virtue of their ability to be made and/or sold separately to be later interfaced with the other components. Moreover, to the extent various of the embodiments provide primarily the ability to provide support scaffolds to AAAs, such embodiments are nevertheless considered “treatment” systems or assemblies to the extent that they provide a mechanism by which AAA support or other treatment may be performed.

Moreover, despite the particular benefits provided by the present embodiments for treating AAAs, they may be suitably modified for application elsewhere or for other indications without substantially departing from the intended broad scope hereof. For example, other forms of aneurysms, such as thoracic aneurysms, cerebral aneurysms, etc., may be treated with suitably modified forms of the embodiments herein shown and described in order to achieve the appropriate delivery into those particular related anatomies, and appropriate support structures and geometries for those particular conditions.

Still further, while these other forms of aneurysms are generally vascular in nature, it is further contemplated that other weakened or aneurismal wall conditions associated with other lumenal structures may also be treated according to suitable modifications of the embodiments hereunder. In still another regard, reference among the various aspects, modes, and embodiments to therapeutic applications to “aneurysms” are also intended to be applicable to other wall conditions that may not be technically aneurysms, but contemplate other weakened or damaged wall conditions that may be for example genetic or otherwise deteriorative conditions.

Still further, the present invention according to its broad aspects is considered to provide highly beneficial lumenal wall therapy in several modes, which are considered beneficial either individually or in their various combinations that may be achieved. For example, progression, or dilation, or both, of aneurysms or other damaged, weakened, or otherwise suspect wall conditions may be reduced according to one or more of the various aspects herein described. In this regard, it is appreciated that progression is reduced either by reducing the rate or risk of a continuing degenerative condition, or by preventing or reducing the risk of a more discrete event, such as in particular for example, but without limitation, rupture of the wall at the treated location.

The present embodiments are highly beneficial in particular regard to external therapies to aneurysms. However, it is further contemplated that various aspects are additionally beneficial when applied in other modes.

Direct injection into wall tissue of certain injectable material embodiments is contemplated, for example.

Moreover, the various aspects applying injectable, molecular, polymeric, living cells, or other biologic materials to aneurysms may be applied either externally, directly into the wall tissue, or internally along the wall. As for the latter case of internal wall coupling of materials, such may be accomplished for example, but without limitation, in conjunction with endograft implantation, such as for example through or behind an endograft wall.

In this regard, the various combinations of mechanical wall scaffolding embodiments together with injectable, molecular, or polymeric material delivery are also contemplated. Still further, cell therapy embodiments have also been herein disclosed, which may be combined with other material delivery embodiments, mechanical or otherwise.

Among the various combinations of the present aspects, modes, and embodiments herein contemplated, it is further appreciated that various aspects may be applied in one manner, such as externally to an aneurysm, whereas other combined aspects may be applied in another manner to the same or related location in the same patient, such as within the wall or along the internal luminal wall surface. Such may be accomplished contemporaneously, or in series in separate steps or procedures according to the particular need and medical setting. In this respect, such aspects may be combined notwithstanding their use in different interventions at distinct times.

According to the various combinations of components and elements herein contemplated, such related tools, devices, and materials may be combined together in kits, or may be provided separately packaged, and in any case may be sold separately or together in a “bundled” fashion.

Moreover, various tools, such as endoscopes, dissection devices, etc. may be used in order to perform the various methods herein described. These may be custom devices for the specific purpose of performing the present invention. Or, they may be otherwise commercially available implements. In any event, to the extent such tools are instructed for use with the present invention, or are otherwise packaged together or sold or promoted in a combined manner with specific embodiments herein described for the present invention, the additional resulting combination(s) are considered further aspects of the invention.

The invention has been discussed in terms of certain particular embodiments. One of skill in the art will recognize that various modifications may be made without departing from the scope of the invention. In addition, while particular cooperating or adjunctive treatment or other accessory devices are described for use in conjunction with the present embodiments, other modifications are contemplated as would be apparent to one of ordinary skill. Moreover, while certain features may be shown or discussed in relation to a particular embodiment, such individual features may be used on the various other embodiments of the invention.

Although the description above contains many details, these should not be construed as limiting the scope of the invention but as merely providing illustrations of some of the presently preferred embodiments of this invention. Therefore, it will be appreciated that the scope of the present invention fully encompasses other embodiments which may become obvious to those skilled in the art, and that the scope of the present invention is accordingly to be limited by nothing other than the appended claims, in which reference to an element in the singular is not intended to mean “one and only one” unless explicitly so stated, but rather “one or more.” All structural, chemical, and functional equivalents to the elements of the above-described preferred embodiment that are known to those of ordinary skill in the art are expressly incorporated herein by reference and are intended to be encompassed by the present claims. Moreover, it is not necessary for a device or method to address each and every problem sought to be solved by the present invention, for it to be encompassed by the present claims. Furthermore, no element, component, or method step in the present disclosure is intended to be dedicated to the public regardless of whether the element, component, or method step is explicitly recited in the claims. No claim element herein is to be construed under the provisions of 35 U.S.C. 112, sixth paragraph, unless the element is expressly recited using the phrase “means for.”

1-5. (canceled)

6. A vascular aneurysm support system, comprising:
a support scaffold agent that is adjustable between a first configuration and a second configuration;
a minimally invasive delivery assembly with a minimally invasive delivery member with a delivery lumen;
means for delivering the support scaffold agent in the first configuration through the delivery lumen from outside a patient's body and to a location within the patient's body externally adjacent to a vascular aneurysm;
wherein the support scaffold agent is adjustable at the location from the first configuration to the second configuration; and
wherein the support scaffold agent in the second configuration at the location is adapted to provide a substantial external support scaffold to the vascular aneurysm against outward pressure from within the vascular aneurysm.

7-12. (canceled)

13. The system of claim 6, wherein:
the support scaffold agent comprises an adjustable mechanical scaffold wall that is adjustable between a first shape in the first configuration and a second shape in the second configuration.

14. The system of claim 13, wherein the adjustable mechanical scaffold wall comprises an adjustable stent scaffold.

15. The system of claim 14, wherein the mechanical scaffold wall further comprises a graft member coupled to the adjustable stent scaffold to form an adjustable stent-graft composite member.

16. The system of claim 13, wherein:
the mechanical scaffold wall comprises a longitudinal axis, first and second opposite longitudinal ends along the longitudinal axis, first and second opposite transverse ends transverse to the longitudinal axis, an adjustable at least partially tubular shape extending at least partially around the longitudinal axis between the transverse ends, and an interior passageway extending between first and second end ports at the first and second longitudinal ends;
the first shape has a first diameter and is deliverable to the location through the delivery lumen;
the second shape comprises a radius of curvature around the longitudinal axis with the first and second transverse ends extending around a substantial portion of a circumference about the longitudinal axis and with a second diameter that is substantially larger than the first diameter;
the first and second transverse ends are relatively adjustable apart from each other so as to form an adjustable lateral opening through a separation between the transverse ends and extending longitudinally between the first and second end ports, and such that the mechanical scaffold wall is adapted to receive the vascular aneurysm of a vessel into the interior passageway laterally through the lateral opening and with the vessel extending through the first and second end ports; and
the mechanical scaffold wall in the second configuration with the second shape is adapted to engage an exterior surface of the vascular aneurysm sufficient to provide external support to the vascular aneurysm against outward pressure from within the vascular aneurysm.

17-22. (canceled)

23. The system of claim 14, wherein the adjustable stent scaffold of the mechanical scaffold wall comprises a nickel-titanium alloy material.

24. The system of claim 13, wherein the mechanical scaffold wall comprises:
a longitudinal axis and a transverse axis; and
a plurality of transverse ribs extending transverse to the longitudinal axis and separated by gaps.

25-38. (canceled)

39. The system of claim 6, wherein the delivery assembly further comprises a laparoscopic delivery assembly with a laparoscope.

40-60. (canceled)

61. The system of claim 6, wherein:
the support scaffold agent comprises a polymer agent;
the first configuration is characterized at least in part by the polymer agent being in a substantially non-polymerized condition; and
the second configuration is characterized at least in part by the polymer agent being in a substantially polymerized condition.

62-78. (canceled)

79. The system of claim 61, further comprising;
a volume of living cells that are adapted to be delivered in combination with the polymer agent through the delivery lumen to the location.

80-87. (canceled)

88. The system of claim 61, wherein the polymer agent comprises an injectable polymer agent.

89. (canceled)

90. The system of claim 6, wherein the support scaffold agent is adapted to provide external scaffold support to an abdominal aortic aneurysm (“AAA”).

91. The system of claim 6, wherein the support scaffold agent is adapted to provide external scaffold support to a cerebral vascular aneurysm.

92. The system of claim 6, wherein the support scaffold agent is adapted to provide external scaffold support to a thoracic aortic aneurysm.

93-101. (canceled)

102. The system of claim 61, wherein:
the polymer agent comprises first and second precursor materials; and
the first and second precursor materials are adapted to polymerize to provide a polymeric scaffold when mixed.

103-109. (canceled)

110. The system of claim 88, wherein the injectable polymer agent comprises first and second precursor agents that are adapted to react when combined at the location.

111-118. (canceled)

119. A method for treating a vascular aneurysm, comprising:
positioning a minimally invasive delivery member with a delivery lumen so as to provide translumenal access from outside a patient's body through the delivery lumen to a location within the patient's body that is externally adjacent to the vascular aneurysm;
delivering a support scaffold agent in a first configuration through the delivery lumen to the location; and
adjusting the support scaffold agent from the first configuration to a second configuration at the location that is adapted to provide a substantial external support scaffold to the vascular aneurysm against outward pressure from within the vascular aneurysm.

120. The method of claim 119, wherein
the support scaffold agent comprises a polymer agent;
the first configuration is characterized at least in part by the polymer agent being in a substantially non-polymerized condition; and
the second configuration is characterized at least in part by the polymer agent being in a substantially polymerized condition.

121. The method of claim 119, wherein
the support scaffold agent comprises living cells.

122. The method of claim 119, wherein:
the support scaffold agent comprises a polymer agent and living cells; and
the living cells and polymer agent are delivered to the external wall of the vascular aneurysm through the delivery lumen at the location together.

123-128. (canceled)
